(19) United States
(12) Patent Application Publication
(10) Pub. No.: US 2021/0321996 A1
(43) Pub. Date: Oct. 21, 2021

(54) DEVICES AND METHODS FOR CATHETER-BASED CARDIAC PROCEDURES

(71) Applicant: MITRX, Inc., San Jose, CA (US)

(72) Inventors: Murali Dharan, Danville, CA (US); Albert K. Chin, Palo Alto, CA (US); John Ashley, Danville, CA (US); Jeffry J. Grainger, Portola Valley, CA (US)

(73) Assignee: MITRX, Inc., San Jose, CA (US)

(21) Appl. No.: 17/364,692

(22) Filed: Jun. 30, 2021

Related U.S. Application Data
(63) Continuation of application No. PCT/US20/13369, filed on Jan. 13, 2020.
(60) Provisional application No. 62/791,510, filed on Jan. 11, 2019.

(51) Int. Cl. A61B 17/00; A61B 6/12; A61B 8/08; A61B 8/12

(52) U.S. Cl. CPC A61B 17/00234; A61B 17/0057; A61B 6/12; A61B 8/0841; A61B 2017/0409; A61B 2017/0034; A61B 2017/00247; A61B 2017/00575; A61B 2017/00663; A61B 8/12

(57) ABSTRACT

Systems, devices, and methods for performing catheter-based procedures in the heart. In specific embodiments a procedural catheter is introduced into the mediastinum from a suprasternal access site. The procedural catheter is passed through a wall of the heart, preferably at an extrapericardial location on the atrial dome. The procedural catheter is used to perform a procedure in the heart such as mitral valve repair or replacement, using remote catheter visualization techniques.

### CROSS-REFERENCE

This application is a continuation of PCT Application No. PCT/US20/13369 (Attorney Docket No. 54513-706.601), filed Jan. 13, 2020, which claims the benefit of U.S. Provisional Application No. 62/791,510 filed Jan. 11, 2019, the entire content of which are incorporated herein by reference.

### TECHNICAL FIELD

This relates generally to devices, systems, and methods for performing catheter-based procedures on or in the heart, including but not limited to, devices, systems, and methods for performing catheter-based procedures on the left atrium and internal structures of the heart.

### BACKGROUND

Heart disease has been the leading cause of death worldwide. Cardiac operations, such as cardiac surgery, cardiovascular surgery, and cardiothoracic surgery, are important (and sometimes the only available) treatment options for many heart diseases.

Traditionally, for cardiac operations, open heart surgeries were performed. Such operations typically involve cutting and opening the chest of a patient (e.g., via a median sternotomy or a thoracotomy approach). Open heart surgery typically includes making a 5-inch to 10-inch incision in the chest, surgical division of the patient's sternum (also called the breastbone), and also sometimes requires prying the rib cage apart. These procedures can be painful and very invasive, and often lead to medical complications which can slow down the recovery of the patient. In addition, patients who are in poor medical condition may not be eligible to receive open heart surgery due to the risks associated with such operations, thereby preventing the much-needed surgical treatment of heart disease.

Minimally-invasive heart surgeries have been developed to reduce the above-discussed issues associated with open heart surgery. In minimally-invasive heart surgeries, smaller incisions (e.g., 1-inch to 4-inch incisions) are made on the chest (e.g., a hemisternotomy incision or a mini-thoracotomy incision made at a location that corresponds to spacing between ribs of a patient, such as an intercostal space).

However, current minimally-invasive techniques often require sawing the sternum (e.g., hemisternotomy) or separating the ribs (e.g., right antero-lateral thoracotomy), which can lead to costochondral disarticulation and rib fractures. Minithoracotomy (e.g., right minithoracotomy), which is performed for mitral valve surgeries, involves making an incision on the chest and opening the pericardium. Video-assisted thoracoscopic (VATS) procedures may also involve placing instruments in the chest cavity between the ribs which can be painful. While less invasive than open heart surgery, even these procedures can be associated with significant complications that are undesirable and may not be tolerated by high risk patients. Further, most such approaches require the use of cardiopulmonary bypass to arrest the heart and lungs, making it possible to operate on a stopped heart. Cardiopulmonary (or heart-lung) bypass has its own significant risks and complications.

In the past two decades, catheter-based approaches for performing valve repair and replacement and other intracardiac procedures have been developed. These involve the introduction of a catheter into a peripheral artery or vein, and advancement of the catheter into the heart, where a prosthesis may be deployed or a repair procedure performed with the heart beating, avoiding the use of cardiopulmonary bypass. Such approaches have achieved widespread success in aortic valve replacement, where a catheter is introduced from a femoral artery into the aorta and a stented valve prosthesis is deployed at the native aortic valve position. In contrast, however, transcatheter approaches to mitral valve replacement or repair have proven far more difficult. Not only is the anatomy of the mitral valve much more complex than the aortic valve, but the endovascular routes to the mitral valve are circuitous and require navigation through tight turns and across the septum of the heart. Achieving the desired repair or replacement using a long, flexible, tightly-curved catheter has proven extremely challenging. Thus, while some simple transcatheter mitral procedures have gained adoption, more complex transcatheter procedures such as mitral replacement, annuloplasty, and chordal replacement are still far away from clinical viability.

In recent years, some surgeons have employed a trans-apical approach to perform mitral valve surgery on the beating heart, which, like transcatheter approaches, can eliminate the need for cardiopulmonary bypass. In this approach, a left mini-thoracotomy is created and an opening is made in the pericardium. An incision is made in the left ventricle of the heart near the apex to place a purse string suture or create a sealed access port through which instruments and/or prostheses can be introduced to perform mitral valve repair or replacement. While the trans-apical approach has the advantage of avoiding cardiopulmonary bypass and further allows the mitral valve to be reached through a much shorter, straighter path than endovascular approaches, it has been found that access through the left ventricle creates significant trauma to this critical left ventricular muscular chamber of the heart and can result in long-term impairment of ejection fraction and/or can cause scar tissue formation in the heart muscle. Further, controlling bleeding from the trans-apical incision is challenging both during and after the procedure due to the high blood pressure in the left ventricle, and the occurrence of bleeding-related complications has been undesirably high. Moreover, this approach requires pericardial access which adds risk and complexity. Therefore, many surgeons and cardiologists believe that the trans-apical approach is not a long-term solution for less-invasive mitral surgery.

Thus, there is a need for systems, methods, and devices that further reduce or eliminate complications associated with cutting, separating, and/or breaking the bones, incising the diaphragm, and/or incising the pericardium, which avoid incisions in the left ventricle, and which allow intra-cardiac procedures to be performed on the beating heart without the need for cardiopulmonary bypass.

### SUMMARY

Some or all of the above deficiencies and other problems associated with conventional cardiac surgical devices and methods may be reduced or eliminated by the disclosed devices and methods.

In a first embodiment, a method of performing an interventional procedure in a beating heart of a patient, the method comprises:
introducing a procedural catheter through a suprasternal penetration at a suprasternal access site into a mediastinum of the patient;
advancing the procedural catheter through the mediastinum to an atrial dome of the heart;
inserting the procedural catheter into a left atrium of the heart through a puncture in the atrial dome while the heart is beating; and
performing an interventional procedure on target tissue in a chamber of the heart with the procedural catheter while visualizing the target tissue using a technique selected from echocardiography, fluoroscopy, and intravascular ultrasound.

In exemplary embodiments, the procedural catheter is advanced through the mediastinum under visualization using a technique selected from echocardiography, fluoroscopy, and intravascular ultrasound.

In exemplary embodiments, the method further comprises placing an endoscopic access device through the suprasternal penetration into the mediastinum, wherein the procedural catheter is advanced through the mediastinum in a working channel of the endoscopic access device.

In exemplary embodiments, the method further comprises positioning an access sheath through the suprasternal penetration into at least a portion of the mediastinum, the procedural catheter being advanced through a lumen of the access sheath.

In exemplary embodiments the access sheath is positioned through the atrial dome into the left atrium, the procedural catheter being advanced through the lumen of the access sheath into the left atrium.

In exemplary embodiments, the interventional procedure is selected from mitral annuloplasty, chordal replacement, or mitral valve replacement.

In exemplary embodiments, the interventional procedure comprises pulmonary vein ablation or atrial ablation.

In exemplary embodiments, the interventional procedure comprises closure or occlusion of the left atrial appendage.

In exemplary embodiments, the method further comprises hemostatically sealing the puncture in the left atrial dome around the procedural catheter while performing the interventional procedure.

In another embodiment, the invention includes a method of performing an interventional procedure in a beating heart of a patient, the method comprising:
introducing an access catheter through a penetration at a suprasternal access site into a mediastinum of the patient;
advancing the access catheter through the mediastinum to an atrial dome of the heart with a sternum and ribs of the patient remaining intact;
advancing a tubular needle from an inner lumen of the access catheter to penetrate the atrial dome and extend into a left atrium of the heart;
inserting a guidewire through the needle into the left atrium; removing the needle from the left atrium while leaving the guidewire extending through the inner lumen into the left atrium;
slidably advancing the access catheter over the guidewire into the left atrium;
removing the guidewire from the left atrium and the access catheter;
inserting a procedural catheter through the inner lumen of the access catheter into the left atrium; and
performing an interventional procedure on target tissue in a chamber of the heart with the procedural catheter, wherein the heart remains beating during the interventional procedure.

In exemplary embodiments, a tubular dilator is positioned in the inner lumen of the access catheter as it is slidably advanced into the left atrium with the guidewire extending through the dilator.

In exemplary embodiments, the interventional procedure is performed under visualization using a technique selected from echocardiography, fluoroscopy, and intravascular ultrasound.

In exemplary embodiments, the access catheter is advanced through the mediastinum using a technique selected from echocardiography, fluoroscopy, and intravascular ultrasound.

In exemplary embodiments, the interventional procedure is selected from mitral annuloplasty, chordal replacement, or mitral valve replacement.

In exemplary embodiments, the interventional procedure comprises pulmonary vein ablation or atrial ablation.

In exemplary embodiments, the interventional procedure comprises closure or occlusion of the left atrial appendage.

In exemplary embodiments, the method further comprises hemostatically sealing the puncture in the left atrial dome around the access catheter while performing the interventional procedure.

In exemplary embodiments, the method further comprises closing the puncture in the left atrial dome after the interventional procedure is performed.

In exemplary embodiments, closing the puncture comprises delivering a suture through tissue of the atrial dome with a closure device positioned through the access catheter.

In still other embodiments, the invention provides a system for performing an interventional procedure in a heart of a patient, comprising:
a mediastinal access device positionable through a suprasternal penetration at a suprasternal access site and configured for advancement through a mediastinum of the patient to a location proximate to an atrial dome of the heart, the mediastinal access device having a working channel therein;
an atrial access catheter slidably positionable through the working channel and having a distal end configured for introduction through a puncture in the atrial dome into a left atrium of the heart with a proximal end thereof extending out the mediastinum through the suprasternal penetration, the atrial access catheter having an inner lumen; and
a procedural catheter positionable in the inner lumen of the atrial access catheter, the procedural catheter having an interventional mechanism in a distal portion thereof configured for performing an interventional procedure in the heart;
wherein the atrial access catheter and the procedural catheter are configured for being visualized in the heart using a technique selected from echocardiography, fluoroscopy, and intravascular ultrasound.

In exemplary embodiments, the system further comprises a tissue penetration device removably positionable in the inner lumen of the atrial access catheter and having a distal tip extendable therefrom configured to penetrate tissue of the atrial dome.

In exemplary embodiments, the tissue penetration device comprises a tubular needle.

In exemplary embodiments, the system further comprises a guidewire slidably positionable through the tubular needle.

In exemplary embodiments, the system further comprises a dilator removably positionable in the inner lumen of the atrial access catheter, the dilator having a passage therein configured to receive the guidewire.

In exemplary embodiments, the mediastinal access device comprises an imaging device for imaging the mediastinum.

In exemplary embodiments, the imaging device comprises an image sensor, optical channel, or a lens.

In exemplary embodiments, the procedural device comprises a mitral valve repair device.

In exemplary embodiments, the mitral valve repair device is configured to deliver a replacement chord.

In exemplary embodiments, the mitral valve repair device is configured to deliver and annuloplasty ring or band.

In exemplary embodiments, the procedural device comprises an ablation device.

In exemplary embodiments, the procedural device comprises a left atrial appendage occlusion or closure device.

In exemplary embodiments, the system further comprises a closure device positionable in the inner lumen of the atrial access catheter and configured to deliver a closure element for closing the puncture upon removal of the atrial access catheter.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

The novel features of the present disclosures are set forth with particularity in the appended claims. For a better understanding of the features and advantages of the various described implementations, in which the principles of the present disclosure are utilized, reference should be made to the Detailed Description below, in conjunction with the following drawings in which like reference numerals refer to corresponding parts throughout the figures.

FIG. 1 is a schematic diagram illustrating the mediastinum in a chest of a patient from the anterior view with overlying skin and muscle removed.

FIG. 2 is a schematic diagram illustrating a location of a suprasternal access site from the anterior view.

FIGS. 3A-3C are flow charts illustrating various embodiments of catheter-based interventional procedures in the heart according to the invention.

FIG. 4A is a schematic diagram illustrating insertion of a percutaneous device through an opening at a suprasternal access site in accordance with some embodiments from a cut-away side view.

FIGS. 4B-4F illustrate delivery of a percutaneous device through the mediastinum to the heart, in accordance with some embodiments from a sagittal view.

FIGS. 5A-5J are schematic diagrams illustrating an exemplary procedure for catheter-based mitral annuloplasty in accordance with some embodiments.

FIG. 6 shows a superior view of a patient's thorax showing a delivery route for a percutaneous device, in accordance with some embodiments.

FIGS. 7A-7C show an extrapericardial location of the roof of the left atrium of the heart, in accordance with some embodiments.

FIGS. 8A-8D illustrate a catheter-based procedure for mitral chordal replacement in accordance with some embodiments.

FIGS. 9A-9B are side views of a leaflet anchor for a replacement chord in accordance with some embodiments.

FIGS. 9C-9D are end views of a leaflet anchors for a replacement chord in accordance with some embodiments.

FIG. 9E is a side cut-away view of a distal portion of a leaflet anchor delivery catheter in accordance with some embodiments.

FIGS. 9F-9H are side views showing the delivery of a leaflet anchor through a mitral leaflet in accordance with some embodiments.

FIG. 9I is perspective view of a leaflet anchor delivery catheter in accordance with some embodiments.

FIG. 10A is a side view of a papillary anchor delivery catheter in accordance with some embodiments.

FIG. 10B is a side cut-away view of a distal portion of the papillary anchor delivery catheter of FIG. 10A.

FIGS. 11A-11C are schematic illustrations of a catheter-based valve replacement procedure, in accordance with some embodiments.

FIGS. 12A, 12C, and 12F are side cut-away views of a closure device for closing a penetration in a heart wall, in accordance with some embodiments.

FIG. 12B is a perspective view of a needle arm and needle tip in the closure device of FIG. 12A.

FIG. 12D is a perspective view of a needle catcher in the closure device of FIG. 12A.

FIG. 12E is a side cut-away view of a distal portion of the closure device of FIG. 12A.

FIGS. 12G-12H are side views of a distal portion of the closure device of FIG. 12A in a heart wall.

Like reference numerals refer to corresponding parts throughout the several views of the drawings. Drawings are not necessarily drawn to scale unless explicitly indicated otherwise.

### DETAILED DESCRIPTION

Reference will now be made in detail to implementations, examples of which are illustrated in the accompanying drawings. In the following detailed description, numerous specific details are set forth in order to provide a thorough understanding of the various described implementations. However, it will be apparent to one of ordinary skill in the art that the various described implementations may be practiced without these specific details. In other instances, well-known methods, procedures, components, circuits, and networks have not been described in detail so as not to unnecessarily obscure aspects of the implementations.

Many modifications and variations of this disclosure can be made without departing from its spirit and scope, as will be apparent to those skilled in the art. The specific implementations described herein are offered by way of example only, and the disclosure is to be limited only by the terms of the appended claims, along with the full scope of equivalents to which such claims are entitled. It will be readily understood that the aspects of the present disclosure, as generally described herein, and illustrated in the figures, can be arranged, substituted, combined, separated, and designed in a wide variety of different configurations, all of which are explicitly contemplated herein.

Although certain embodiments and examples are disclosed below, inventive subject matter extends beyond the specifically disclosed embodiments to other alternative embodiments and/or uses, and to modifications and equivalents thereof. Thus, the scope of the claims appended hereto is not limited by any of the particular embodiments described below. For example, in any method or process disclosed herein, the acts or operations of the method or process may be performed in any suitable sequence and are not necessarily limited to any particular disclosed sequence. Various operations may be described as multiple discrete operations in turn, in a manner that may be helpful in understanding certain embodiments, however, the order of description should not be construed to imply that these operations are order dependent. Additionally, the structures, systems, and/or devices described herein may be embodied as integrated components or as separate components.

For purposes of comparing various embodiments, certain aspects and advantages of these embodiments are described. Not necessarily all such aspects or advantages are achieved by any particular embodiment. Thus, for example, various embodiments may be carried out in a manner that achieves or optimizes one advantage or group of advantages as taught herein without necessarily achieving other aspects or advantages as may also be taught or suggested herein.

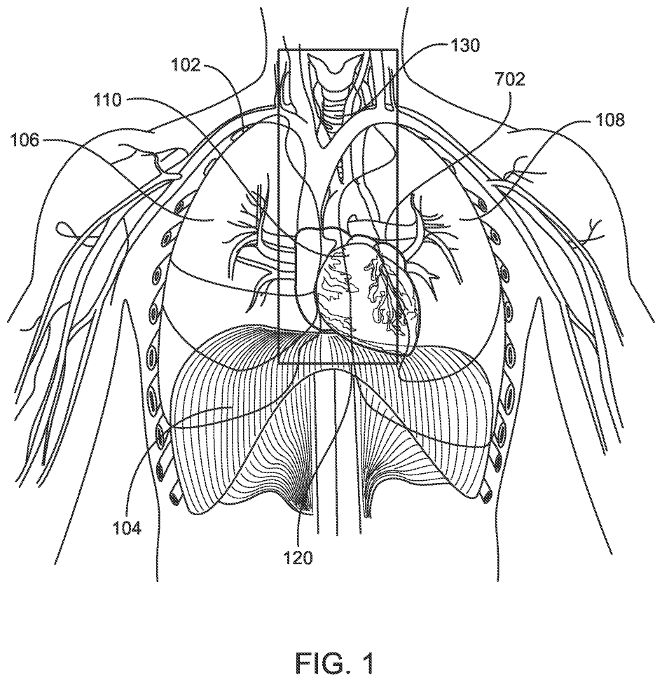

FIG. 1A is a schematic diagram illustrating an example thoracic cavity. A thoracic cavity (also called a chest cavity) is a chamber of a body of a patient that is surrounded by a rib cage 102 and has a thoracic diaphragm 104 at the bottom. Multiple vital organs, such as lungs 106, 108 and the heart 110, are located within the thoracic cavity. Because the breast bone (i.e. sternum, see e.g. FIG. 6), rib cage 102, and the thoracic diaphragm 104 protect the thoracic cavity, conventional heart surgery techniques require cutting, removing, and/or separating one or more portions of the sternum, rib cage 102, and/or the thoracic diaphragm 104 to gain access to the heart 110 (e.g., for delivery of percutaneous devices to the heart).

The mediastinum 120 is a central compartment of the thoracic cavity located between the lungs outside the pleural cavities, and which includes the heart, trachea, esophagus, and other major vessels. As outlined generally in FIG. 1, the mediastinum 120 is located between the lungs 106, 108, and a heart 110 and trachea 130 are located within the mediastinum 120 (e.g., in the middle mediastinum). In some embodiments, the middle mediastinum 120 is accessed through the superior mediastinum (e.g., a portion of the mediastinum from the thoracic inlet to the area above the line from the sterno-manubrial junction to the 4^{th} thoracic vertebra). The mediastinum 120 may be located between the right and left pleural cavities which surround the right and left lungs 106, 108, respectively.

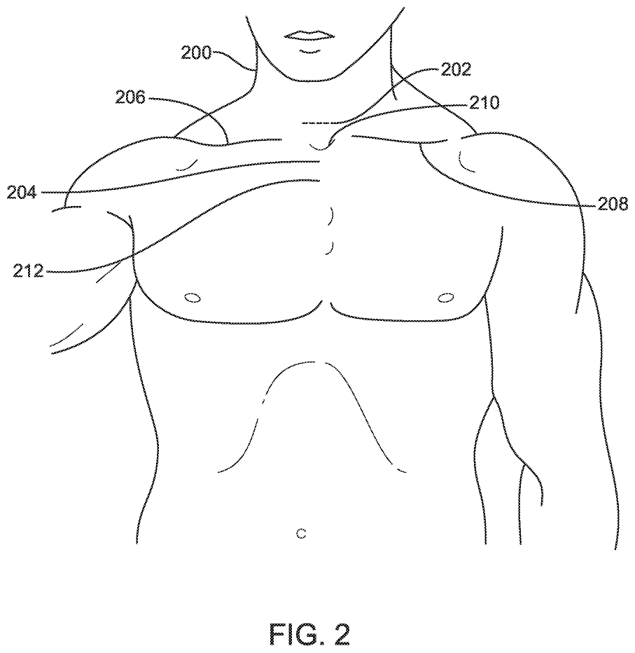

In some embodiments, the mediastinum 120 (e.g., the middle mediastinum) is accessed through the superior thoracic aperture (see e.g. FIG. 2), thereby eliminating the need for cutting, removing, or separating one or more portions of the sternum (see e.g. FIG. 6), the rib cage 102 and/or the thoracic diaphragm 104. This, in turn, reduces or eliminates the problems associated with such procedures.

FIG. 2 is a schematic diagram illustrating a location for accessing the mediastinum accordance with some embodiments. In some embodiments, the access location is in a neck region 200 of a patient superior to the sternum 212 (e.g., at penetration site 202). Penetration site 202 may be located, for example, in the suprasternal notch 210 which is the triangular space at the superior border of the manubrium of the sternum between the clavicular notches 204, 206. Percutaneous catheters and other devices are delivered through the suprasternal notch and mediastinum toward the heart of the patient. This method of delivering devices and catheters through the mediastinum (e.g., the superior mediastinum) is called herein a mediastinal approach.

“Percutaneous catheters” or “percutaneous devices” as used in this disclosure are intended to mean diagnostic, visualization, or interventional catheters and other devices which are adapted to penetrate the skin and underlying tissues to gain access to a body cavity, organ, or vessel through a needle puncture or a very small penetration or incision. Usually the percutaneous catheters and devices will be of small profile, e.g. less than about 12 mm in diameter, and will be flexible and often steerable to allow the devices to be controlled from outside the body to access target structures on or within the heart while avoiding non-target vessels and other anatomical structures, typically requiring indirect visualization techniques such as fluoroscopy, ultrasound, or endoscopy. Conventional percutaneous techniques such as the Seldinger technique may be used to introduce the percutaneous catheters and devices of the invention into the mediastinum or other body cavities and lumens, but are not required.

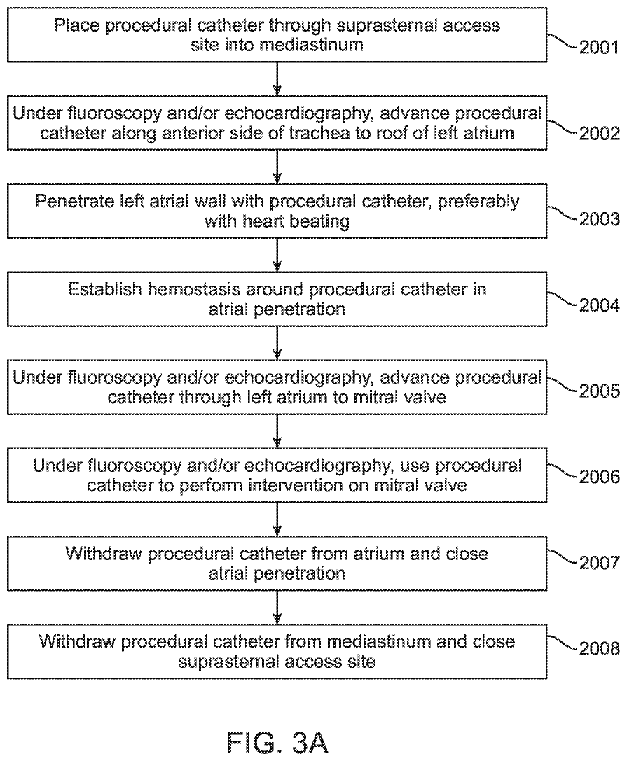

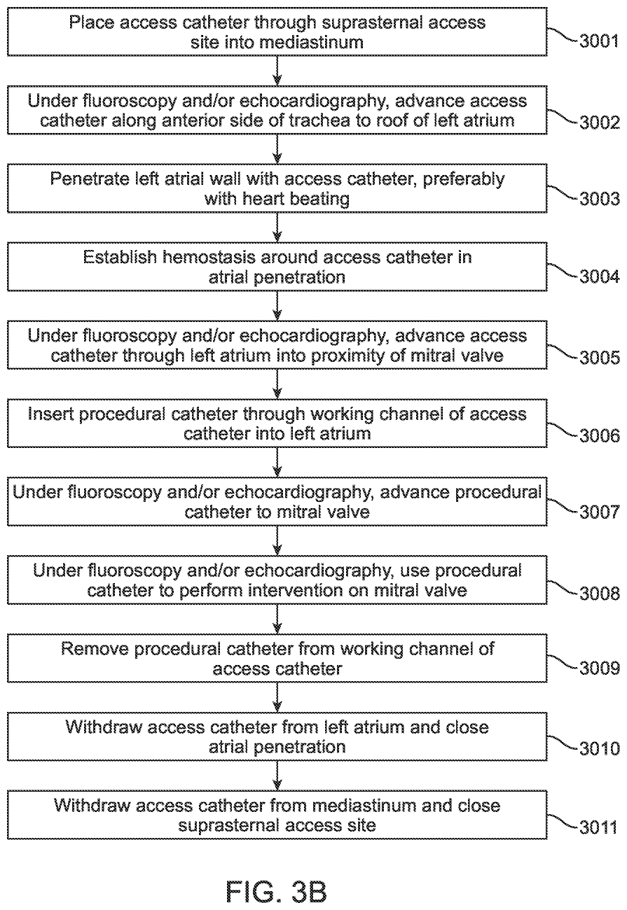

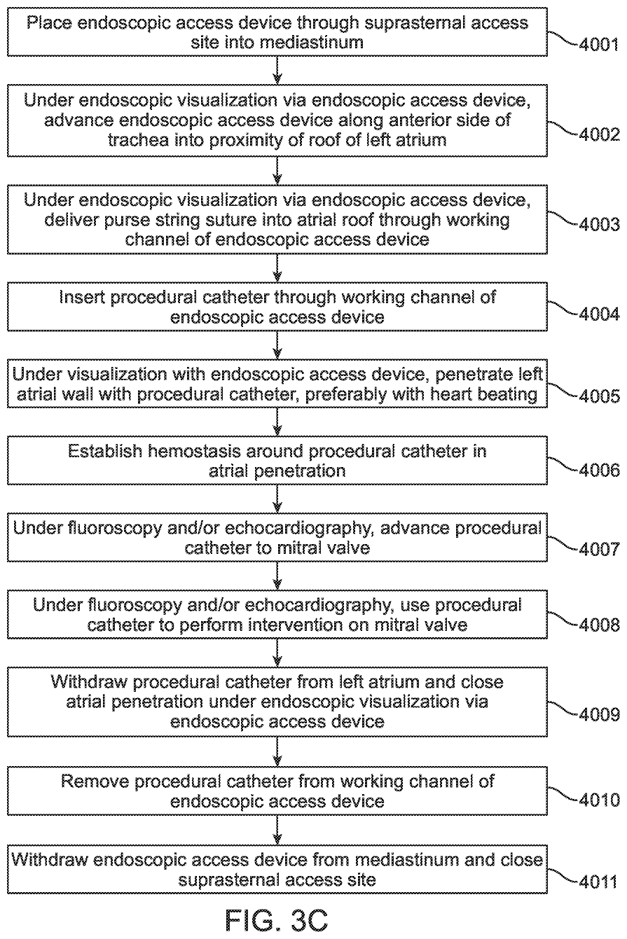

FIGS. 3A-3C are flowcharts summarizing embodiments of exemplary percutaneous mitral valve procedures according to the invention. These methods and the devices used therein are more fully described below.

In the embodiment of FIG. 3A, a small incision or puncture is made at a suprasternal location such as the suprasternal notch to create a suprasternal access site. A procedural catheter is placed through the suprasternal access site into a mediastinum of the patient (Step 2001). The procedural catheter is then advanced inferiorly along the anterior side of the trachea, visualizing the catheter using fluoroscopy or echocardiography (Step 2002). The procedural catheter is advanced to the atrial roof (or dome), preferably to an extrapericardial location on the atrial roof (as further described below). A penetration or puncture is made through the atrial wall and the procedural catheter inserted through the penetration into the left atrium (Step 2003). Hemostasis is then established around the procedural catheter to prevent loss of blood from the heart (Step 2004). Under visualization using fluoroscopy and/or echocardiography, the procedural catheter is positioned at or near the mitral valve (Step 2005). The precise location will depend upon the procedure being performed. If the procedure is mitral annuloplasty, the procedural catheter will be positioned at the mitral annulus, while for chordal replacement, the procedural catheter will be positioned near the edges of the mitral leaflets and/or near the papillary muscles in the left ventricle. The procedural catheter is then used to perform an intervention such as mitral valve repair (Step 2006). When the procedure is complete, the procedural catheter is withdrawn from the left atrium and the atrial penetration is closed (Step 2007). Finally, the procedural catheter is withdrawn from the mediastinum and the suprasternal access site is closed (Step 2008).

It will be understood that there may be variations in the procedure. For example, the procedural catheter may be slidably advanced over a guidewire or a small diameter endoscope inserted through the suprasternal access site to assist in navigating through the mediastinum or in the heart, as described in more detail below. Alternatively, the procedural catheter may be inserted through the working channel of an endoscope or mediastinoscope which has first been advanced through the mediastinum to a position near the left atrial roof

In the embodiment of FIG. 3B, a percutaneous access device, i.e. a catheter, cannula or sheath, is first inserted through a suprasternal access site into the mediastinum (Step 3001) and advanced through the mediastinum under fluoroscopy or echocardiography to the left atrial dome (Step 3002). In a first variation of the procedure, the access device is then advanced through a penetration or puncture in the atrial wall to enter the left atrium, still under visualization with fluoroscopy or TEE (Step 3003). In a second variation, the access device is advanced to a point just above the left atrial dome, and a separate access sheath is inserted through a channel of the access device and advanced through the atrial roof into the left atrium while viewing with TEE or fluoroscopy. In either variation, hemostasis is established around the access device or access sheath to inhibit blood loss from the left atrium through the penetration, in some cases by placing a purse string suture around the site of the atrial penetration, either before or after placement of the access device or sheath (Step 3004). Optionally the access device or sheath is steerable and may be advanced to a position in proximity to the mitral valve or other tissue targeted for intervention while being visualized under fluoroscopy or echocardiography (Step 3005). A procedural catheter is then inserted through a channel of the access device or sheath so as to enter the left atrium (Step 3006). The procedural catheter is preferably steerable and is advanced to the location where the intervention, e.g. mitral valve repair, is to be performed, again visualizing the device using fluoroscopy and/or echocardiography (Step 3007). The procedural catheter is then used to carry out the intervention, such as mitral annuloplasty or chordal replacement (Step 3008). Once complete, the procedural catheter may be removed from the channel of the access device or sheath (Step 3009) and the access device may be withdrawn from the left atrium (Step 3010). The atrial penetration is closed (Step 3010) and the access device is withdrawn from the mediastinum, finally closing the suprasternal access site (Step 3011).

In a third embodiment, summarized in the flowchart of FIG. 3C, an endoscopic access device is first positioned into the mediastinum through the suprasternal access site (Step 4001). The endoscopic access device may include a channel for receiving an endoscope, or may have an image sensor such as a CMOS or CCD chip or other suitable imaging means mounted near its distal end to allow viewing of the field distally and laterally of the distal tip. Under endoscopic visualization, endoscopic access device is advanced through the mediastinum along the trachea until the tip is in proximity to the atrial roof (Step 4002). A suture placement instrument is then inserted through a working channel of the endoscopic access device to place a purse string suture (or other suitable means for sealing around a catheter) in the atrial roof around the site where the atrium is to be entered (Step 4003). Preferably this site will be an extrapericardial location on the atrial roof as described further below. The suture placement device is removed, and a procedural catheter is inserted through the working channel of the endoscopic access device (Step 4004). While viewing the atrial roof with the endoscopic access device, the procedural catheter is advanced through a penetration in the atrial wall to enter the left atrium (Step 4005). The purse string suture may be then tightened to seal around the procedural catheter, establishing hemostasis (Step 4006). Visualizing the interior of the heart with fluoroscopy or echocardiography, the procedural catheter is advanced to the target site (Step 4007) and is used to perform the desired intervention on the mitral valve (Step 4008). Following the repair, the procedural catheter may be withdrawn from the left atrium (Step 4009) and removed from the endoscopic access device (Step 4010). Another device may optionally be inserted through the working channel to secure the purse string suture and seal the penetration. The endoscopic access device may then be withdrawn from the mediastinum and the suprasternal access site closed (Step 4011).

In a variation on the procedure outlined in FIG. 3C, after the endoscopic access device has been positioned in proximity to the atrial dome, an access sheath may be placed through the endoscopic access device and introduced through a puncture in the atrial roof into the atrium. The introduction into the atrium may be visualized endoscopically with an imaging device on, or positioned through, the endoscopic access device. Once within the atrium, the access sheath may be visualized with fluoroscopy or TEE. A procedural catheter may then be inserted through the access sheath into the left atrium to perform an interventional procedure such as valve repair or replacement. In some embodiments the access sheath will be steerable and dimensioned to reach to a point near the tissue of interest, e.g. the mitral annulus, leaflet, or papillary muscle, so that it can be used to guide the procedural catheter to the target tissue and to stabilize the procedural catheter while it performs the intervention.

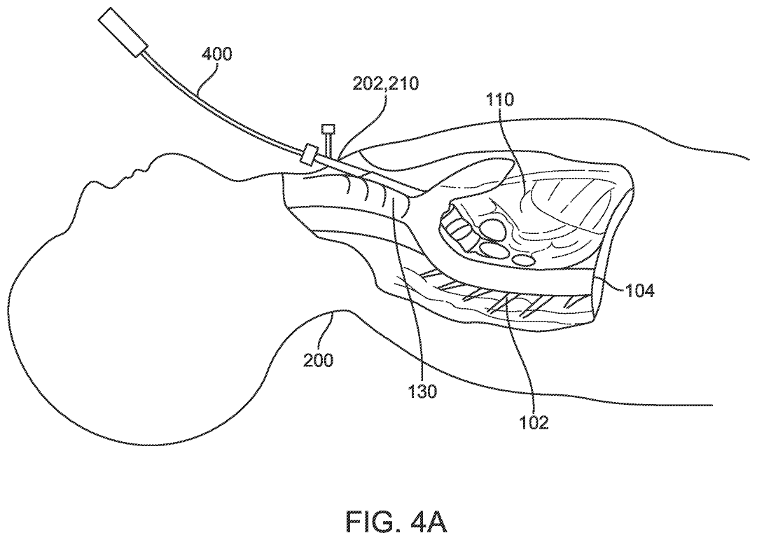

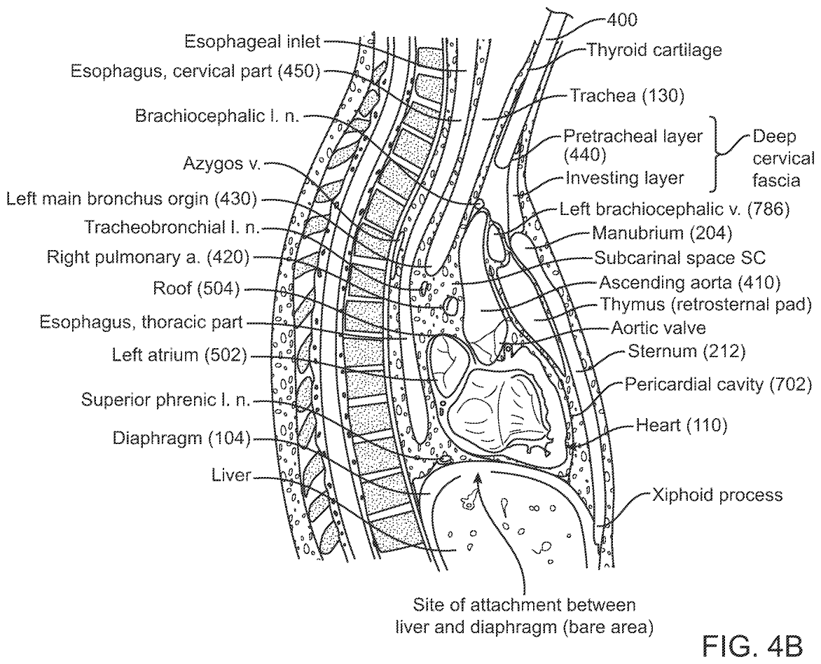

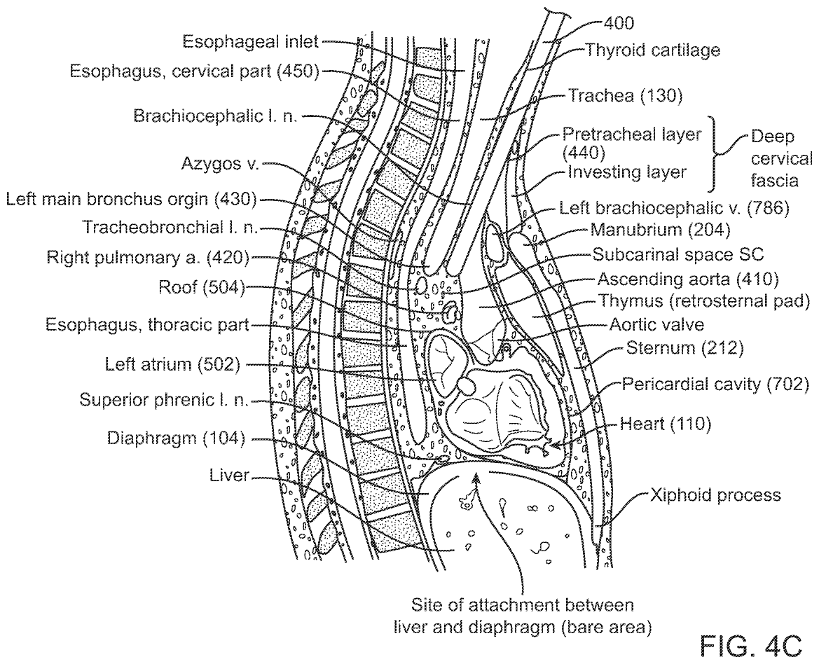

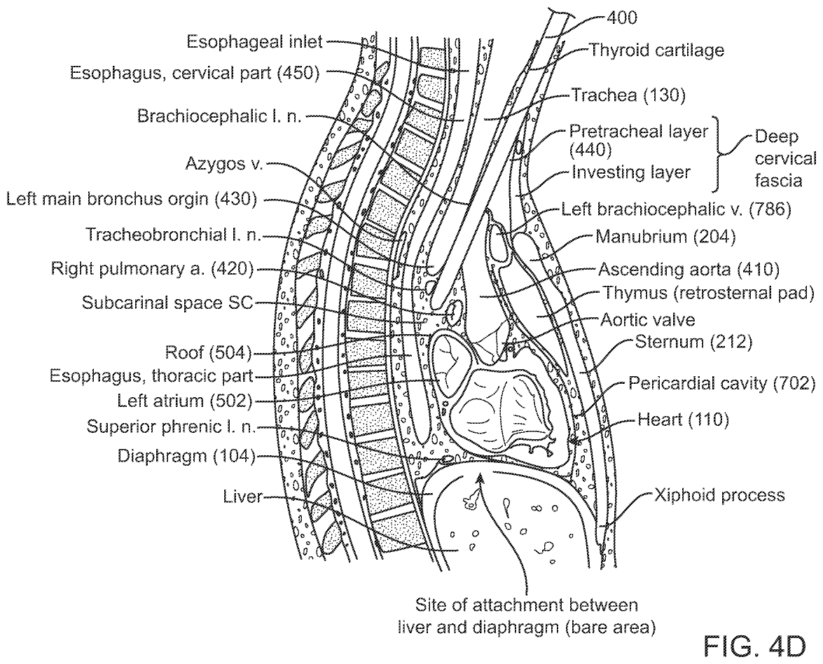

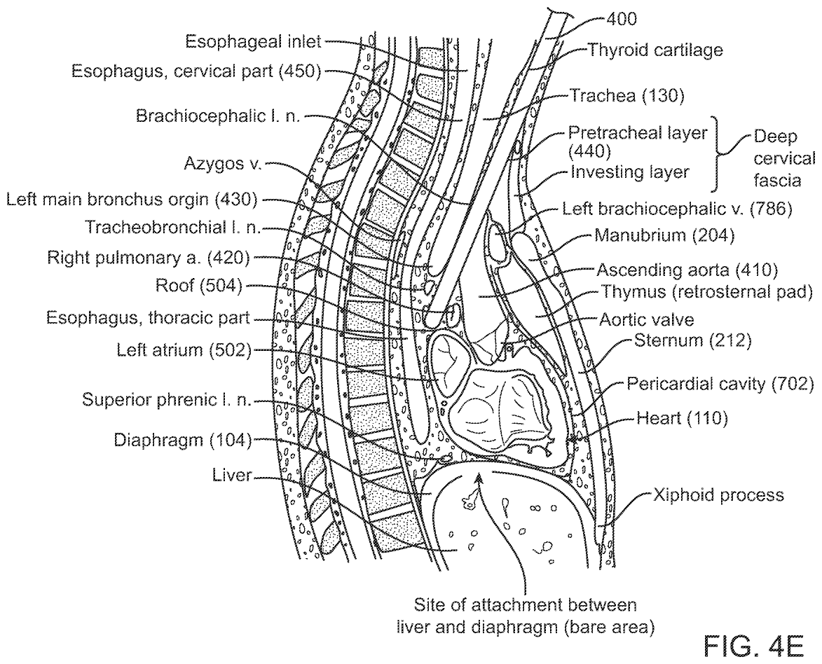

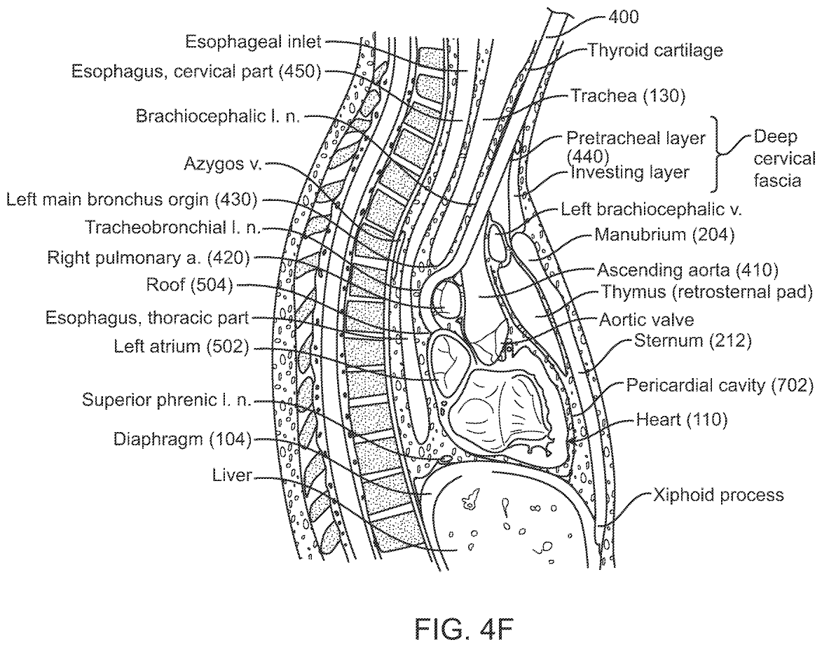

Referring now to FIGS. 4A-4F, percutaneous methods and devices for accessing the interior of the heart according to the invention will be described in greater detail. FIG. 4A is a schematic diagram illustrating insertion of a percutaneous device 400 introduced through an introducer sheath 210, which is placed in an opening 202 in or adjacent to a patient's neck 200 in accordance with some embodiments. In preferred embodiments, the opening 202 is located in or near the suprasternal notch. The introducer sheath 210 is inserted through the opening 202, which allows direct access to the mediastinum without having to cut, open, remove, and/or separate the sternum or the rib cage 102 and/or the thoracic diaphragm 104. Introducer sheath 210 is adapted to be introduced through the skin via a needle puncture or other small penetration, using e.g. the Seldinger technique. Once introducer sheath 210 is in place, percutaneous device 400 may be introduced into the mediastinum through introducer sheath 210.

Introducer sheath 210 may optionally include a port or lumen (not shown) through which a gas such as carbon dioxide may be delivered into the mediastinum to inhibit air from entering the cavity. Introducer sheath 210 may also include a fluidic valve through which percutaneous device 400 may be introduced and which seals around the device to inhibit loss of gas, blood, or other fluids from the mediastinum.

Introducer sheath 210 may optionally be shaped or shapable into a curve or angle to direct percutaneous device 400 in the caudal or inferior direction parallel to the trachea. For example an axis of a distal extremity of introducer sheath 210 may be disposed at an angle of about 90-160° relative to the axis of a proximal extremity of the introducer sheath. Introducer sheath 210 may optionally have a region which is flexible or shapable to allow the user to adjust the relative angle of the distal and proximal extremities in situ.

FIG. 6 shows an exemplary delivery route for a percutaneous device to access the heart or great vessels (e.g. an intracardiac access device as described herein). The distal portion of the percutaneous device may be advanced into the body of the patient along the trachea through the superior thoracic aperture 602 (also referred to herein as the superior thoracic inlet) of the patient. The superior thoracic inlet 602 is the opening at the top of the thoracic cavity surrounded by a bony ring just below (i.e. inferior to) the neck. Insertion of the distal portion of the device through the incision in the suprasternal notch, and subsequent advancement of the instrument through the superior thoracic aperture 602 of the patient toward a left atrium of a heart of the patient may allow a user to access the heart of the patient without cutting a bone (e.g. the sternum 212, manubrium 204, or a rib 102) and/or the thoracic diaphragm 104, or spreading the ribs 102, thereby avoiding the complications associated with such injuries as described herein.

As shown in FIGS. 4A-4F, the distal portion of the percutaneous device 400 may be advanced along the trachea 130, through the thoracic aperture, and toward the left atrium of the patient's heart 110. The path along the trachea 130 may for example be a path anterior to the trachea 130. the distal portion of the percutaneous device 400 may be configured to contact the cardiac wall on the dome 504 of the left atrium 502 while the proximal portion extends out of the patient at penetration site 202.

FIGS. 4B-4F are sagittal cross-sections of a chest region of a patient. Shown on the right hand side is the anterior portion of the patient and on the left hand side of FIG. 4A is the posterior portion of the patient, including the spine.

FIG. 4B illustrates insertion of a percutaneous device 400 through the mediastinum, wherein the percutaneous device 400 is inserted and advanced along the trachea 130 (e.g., along a path anterior to the trachea).

FIG. 4C shows that the percutaneous device 400 is advanced further into a space between the trachea 130 and the ascending aorta 410.

In some embodiments, the distal portion of the percutaneous device 400 may be advanced along a path substantially parallel to a plane containing a longitudinal access of the trachea 130.

In some embodiments, the distal portion of the percutaneous device 400 may be advanced along a path that is superficial to the pretracheal fascia 440. In some embodiments, the distal portion of the percutaneous device 400 may be advanced along a path that is deep to the pretracheal fascia 440. Distal dissection in this plane may lead to the subcarinal space SC inferior to the carina and superior to the left atrial dome or roof 504.

FIG. 4D illustrates that the percutaneous device 400 is advanced even further into a space between the trachea 130 and a branch of the pulmonary artery 420 (e.g., a right pulmonary artery). In some embodiments, the distal portion of the percutaneous device 400 may be advanced substantially parallel to a plane defined by a primary bronchus 430.

In some embodiments, the distal portion of the percutaneous device 400 may move (e.g. without puncturing or damaging) the right or left pulmonary artery 420 aside to reach the dome of the left atrium.

FIG. 4E illustrates that the percutaneous device 400 is advanced toward the heart 110 (e.g., the cardiac wall, such as the wall of the left atrium). In some embodiments, the percutaneous device 400 comes in contact with the heart 110 (e.g., the wall of the left atrium). The distance between the carina (the bifurcation of the trachea into the left and right main bronchii) and the dome or the roof of the left atrium may depend on the size of the left atrium. For example, there may be an inverse correlation between the left atrial size and this distance.

Although FIGS. 4C-4E show the percutaneous device 400 located behind the ascending aorta 410 (e.g., behind the innomate artery and the aortic arch) and/or the pulmonary artery 420, the percutaneous device 400 can be passed through the space between the trachea 130 and the ascending aorta 410 and the space between the trachea 130 and the pulmonary artery 420.

The path through which the device traverses to reach the heart from the penetration site may for example have a length within a range of about 5 cm to about 25 cm, for example within a range of about 10 cm to about 25 cm, or within a range of about 5 cm to about 20 cm. For example, the path may be about 15 cm long.

In preferred embodiments, the percutaneous device 400 may be inserted from the suprasternal incision to the left atrium along a path that passes outside the trachea 130, aorta 410, right pulmonary artery 420, pericardium 702, and other vessels and structures within the mediastinum without entering, penetrating, cutting, puncturing, or otherwise injuring such vessels and structures (other than the left atrium). In some embodiments, the percutaneous device 400 may be introduced into the patient without accessing or penetrating the pleural cavities surrounding the lungs. By not penetrating the pleural cavities, the percutaneous device may avoid pneumothorax which can attend other approaches.

In preferred embodiments, at least a portion of the percutaneous device 400 is flexible and can bend into one or more curves along its length so as to extend around vessels and other structures disposed between the suprasternal access point and the left atrium. Percutaneous device 400 may further have a steerable or articulated distal portion to avoid one or more internal structures of the patient such as the trachea 130, esophagus 450, aorta 410, superior vena cava 720, aortic arch 780, carotid artery 782, innominate artery, left recurrent laryngeal nerve, pulmonary artery 420 and/or a primary bronchus 430 of the patient. Such steerability and/or articulation may also allow the percutaneous device to be positioned on or within the heart 110 at a desired location or angle. In some embodiments, an obturator is removably positionable within a channel of the percutaneous device to straighten and/or stiffen the device during introduction, e.g. to pass between tight anatomical structures or to bluntly dissect tissue.

The percutaneous device 400 may, for example, have a steerable distal end. The distal end may be configured to be steered during advancement to the atrium to align the distal end with a particular access point on the heart, for example an extrapericardial location on the roof of the left atrium. Alternatively or in combination, the distal end may be configured to be steered after being inserted into an internal chamber of the heart, e.g. to align a procedural device or prosthesis at a desired distance and/or angle relative to an internal structure of the heart, for example a mitral valve leaflet, mitral annulus, or papillary muscles, as further described below.

FIG. 4F illustrates a steerable percutaneous device 400, which can be steered by the operator from outside the body to avoid or target particular anatomical structures. As shown in FIG. 4F, in some embodiments, the percutaneous device 400 is steered around the pulmonary artery 420 to reach the left atrium 502. This reduces the extent of displacement, squeezing, and/or bending of the right pulmonary artery 420 in accessing the left atrium 502. Percutaneous device 400 may be steered around other structures and along other routes through the mediastinum depending on patient anatomy and the region of the heart or great vessels targeted for access or treatment.

In some embodiments, the percutaneous device 400 is steered along the coronal plane, in addition to, or instead of, steering along the sagittal plane. For example, as shown in FIG. 1, the heart 110 is located slightly off from the sagittal plane. In some embodiments, the percutaneous device 400 is steered between the left main bronchus 430 and the pulmonary artery 420 to access the left atrium 502. Thus, in some cases, the percutaneous device 400 is also steered toward the left side of the patient to access the left atrium 502. In some embodiments, the percutaneous device 400 is advanced at a diagonal angle from the opening 202 to the left atrium 502.

In some embodiments, the percutaneous device 400 may be configured to fit within a working channel of an endoscopic access device such as an endoscope or mediastinoscope. Such endoscopic access devices are described in commonly assigned PCT Application No. PCT/US18/42171, which has been incorporated herein by reference. The endoscopic access device may be placed through a penetration in the suprasternal notch and advanced into the mediastinum. In some embodiments, the endoscopic access device may be advanced toward the heart 110 in the manner described herein with respect to the percutaneous device 400, e.g. inferiorly along the trachea 130 and between the aorta 410 and the trachea 130 and/or between the right pulmonary artery 420 and the trachea 130. Alternatively, the endoscopic access device may pass behind the aorta 410 and/or right pulmonary artery 420. The endoscopic access device may be advanced until a roof of the left atrium is visible through the optical channel, image sensor (CCD or CMOS chip) or lens of the endoscopic access device. The distal portion of the percutaneous device 400 may then be inserted into a working channel of the endoscopic access device. The distal portion of the percutaneous device 400 may be advanced towards the heart 110 through the working channel of the endoscopic access device. The distal portion of the percutaneous device 400 may be advanced from the distal end of the endoscopic access device to contact the cardiac wall of the heart 110, optionally while being visualized through the optical channel, image sensor, or lens of the endoscopic access device. The endoscopic access device may optionally be used to visualize the mediastinal cavity and/or the heart 110 of the patient while advancing the distal portion of the percutaneous device 400 toward the heart as described herein.

In some embodiments, the percutaneous device 400 may be configured to be slidingly coupled to and advanced over an endoscope. Preferably the endoscope will have a small profile, with a diameter of less than about 10 mm, more preferably a diameter of 5 mm or less, and will be steerable to allow steering around vessels and other structures of the mediastinum. The endoscope may be inserted through the suprasternal penetration and advanced to the left atrium in the manner described above. The structures and vessels of the mediastinum may be viewed with the endoscope while it is advanced to facilitate navigation and minimizing trauma. The endoscope may be advanced until the left atrium can be seen, or until the endoscope reaches the left atrial dome. The percutaneous device 400 may be configured to slidably couple to the endoscope, e.g. by passing the endoscope through a working channel of the percutaneous device 400. The distal portion of the percutaneous device 400 may be slidingly advanced towards the heart over the endoscope.

In some embodiments, the percutaneous device 400 may be configured to be inserted into the opening in the suprasternal notch by being advanced over a guidewire. The guidewire may first be inserted through the opening and advanced through the mediastinum to the left atrium along a path as described above with respect to the percutaneous device 400. In some embodiments, the guidewire may be a steerable guidewire. In some embodiments, the guidewire may be advanced until it contacts the roof of the left atrium of the heart 110. In some embodiments, the tip of the guidewire may be advanced through the roof of the left atrium into the interior of the left atrium. The guidewire may include a pressure transducer, ultrasound transducer, or other sensor (e.g. similar to sensor 530 described herein) configured to detect the location of the guidewire. The guidewire may include radiopaque materials or markers which can be seen using fluoroscopy to assist in navigation. The percutaneous device 400 may include a guidewire lumen, eyelet, tube, or the like configured to be slidably coupled to the guidewire. Alternatively, the guidewire may be passed through a working channel of the percutaneous device 400. The distal portion of the percutaneous device 400 may be advanced towards the heart 110 by sliding over the guidewire.

Various visualization techniques may be used to visualize the percutaneous devices of the invention within the mediastinum and heart. In some embodiments, percutaneous device 400 includes radiopaque markers at certain locations in the distal portion thereof to facilitate visualization using fluoroscopy. Radiopaque dyes or fillers may also be included in the materials used to construct percutaneous device 400. Further, fluoroscopic navigation aids may be used in conjunction with percutaneous device 400, such as the use of radiopaque markers on a tracheal tube placed in the patients' trachea during the procedure. In this way the position of markers on the percutaneous device 400 may be viewed relative to the tracheal tube markers to establish its position in the mediastinum. Similarly, an esophageal tube with markers could be placed in the esophagus during the procedure.

Additionally or alternatively, echocardiography may be used for visualization. Transesophageal echocardiography (TEE) can be used for visualization of the percutaneous device in the mediastinum inferior to the trachea as well as in the heart. An ultrasound transducer may also be placed in the trachea, e.g. incorporated into a tracheal tube, to allow echocardiographic visualization of the percutaneous device as it is advanced along the anterior side of the trachea. Three dimensional echocardiography is particularly preferred for highly detailed visualization. Moreover, the percutaneous device itself may include an ultrasound transducer in the distal tip thereof similar to an intravascular ultrasound (IVUS) catheter to allow ultrasonic visualization as the device is inserted.

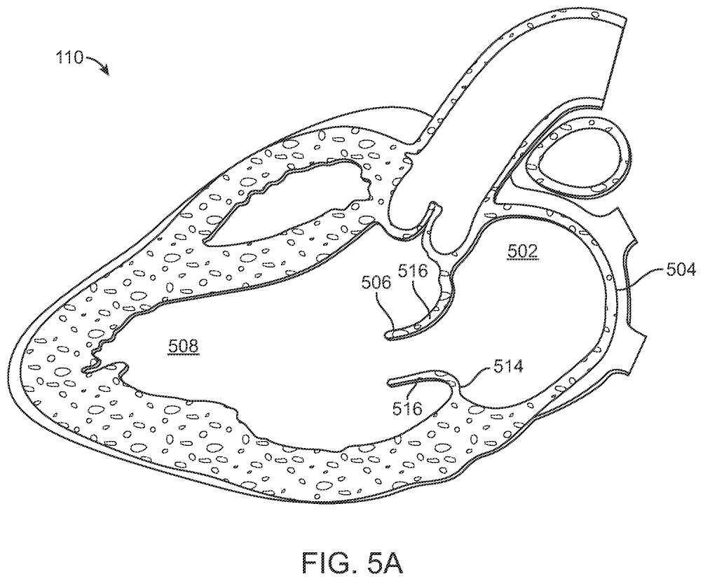

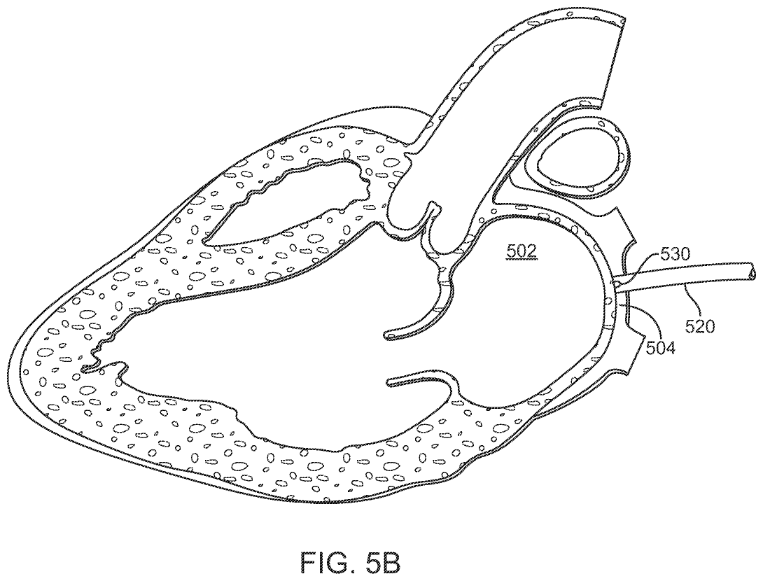

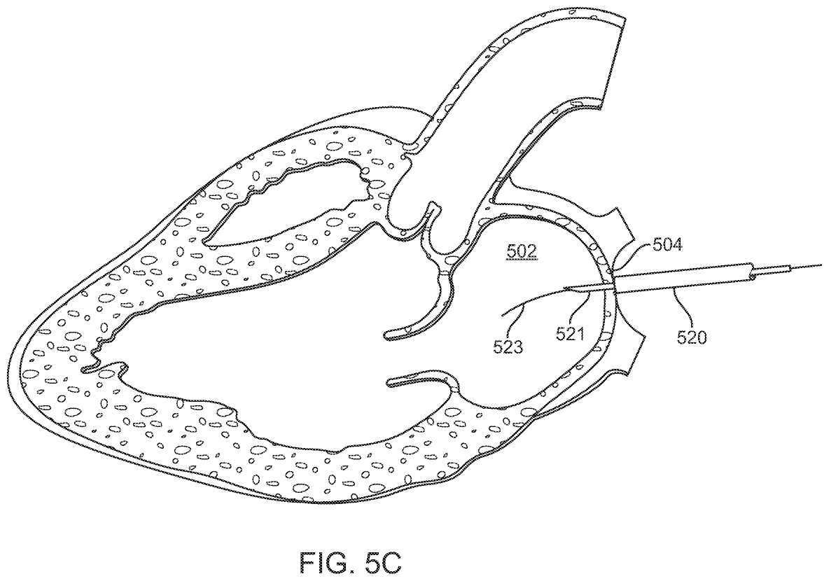

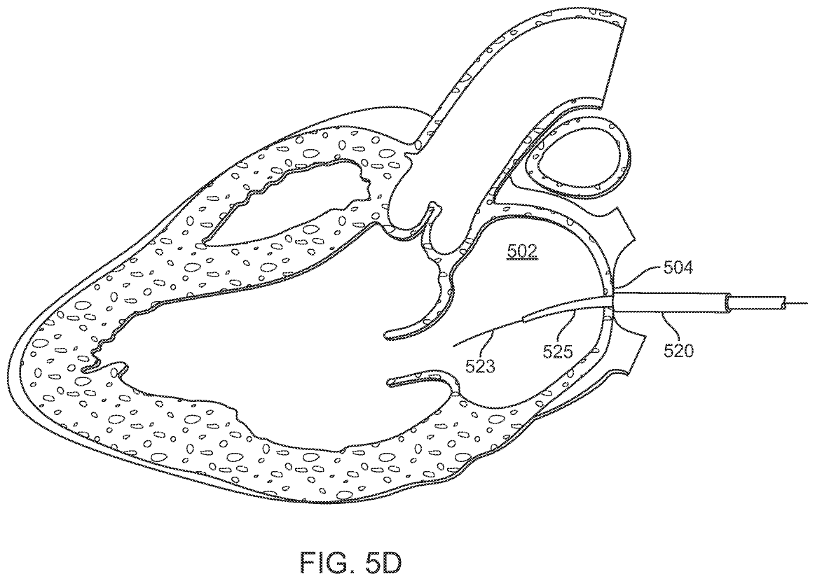

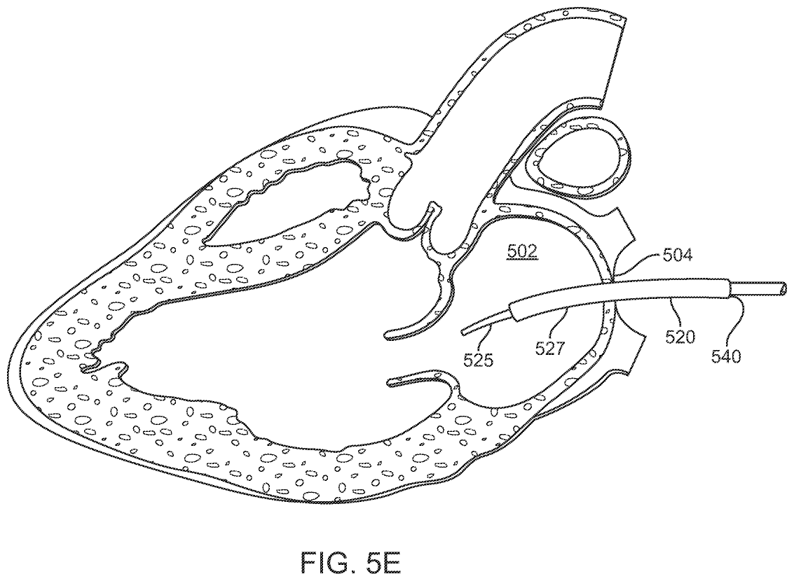

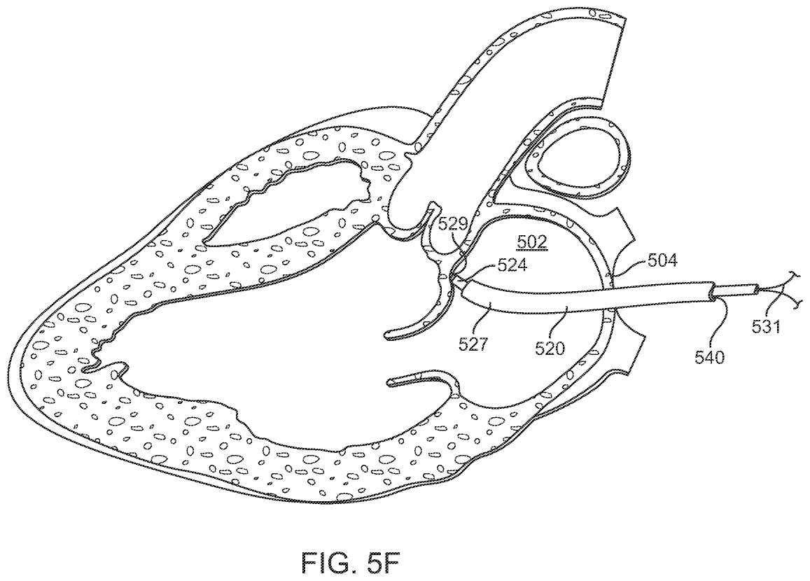

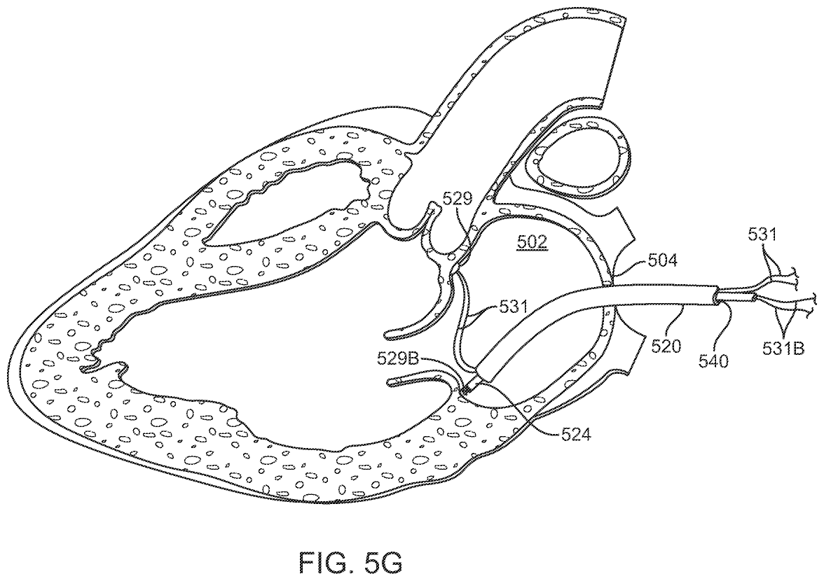

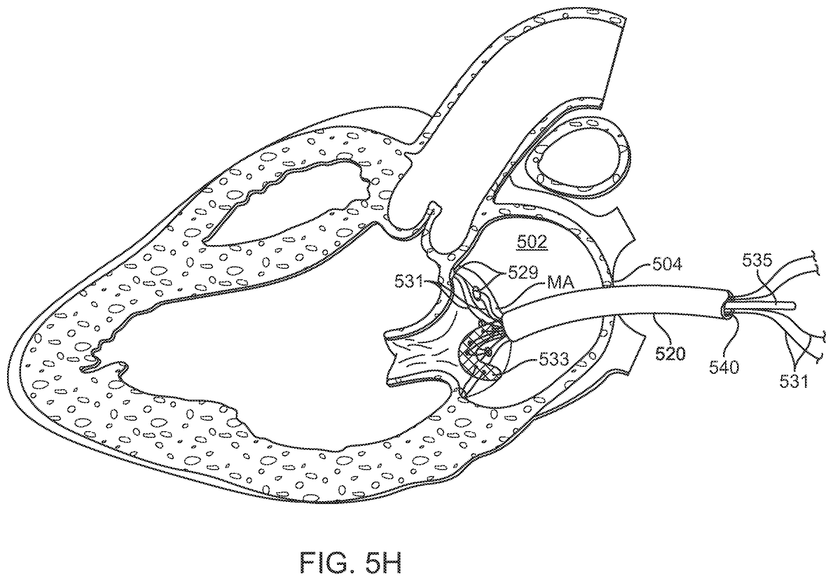

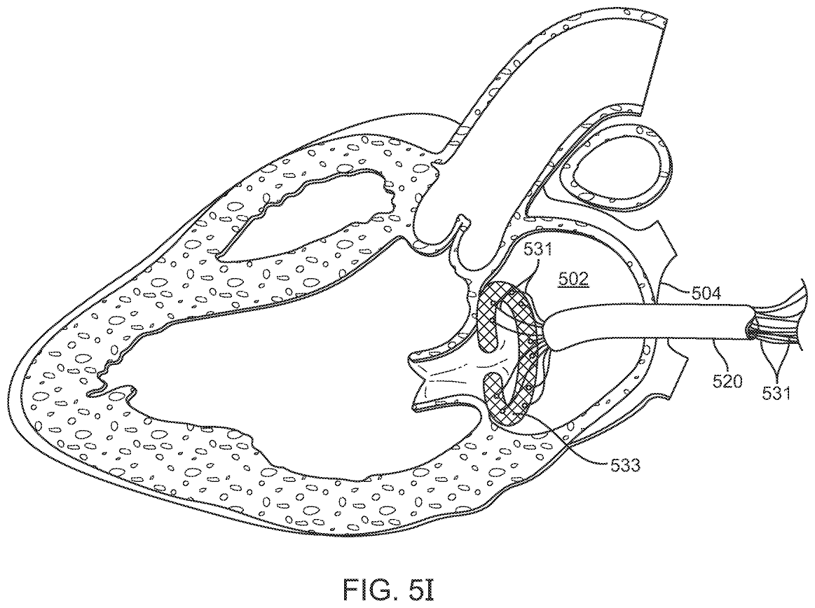

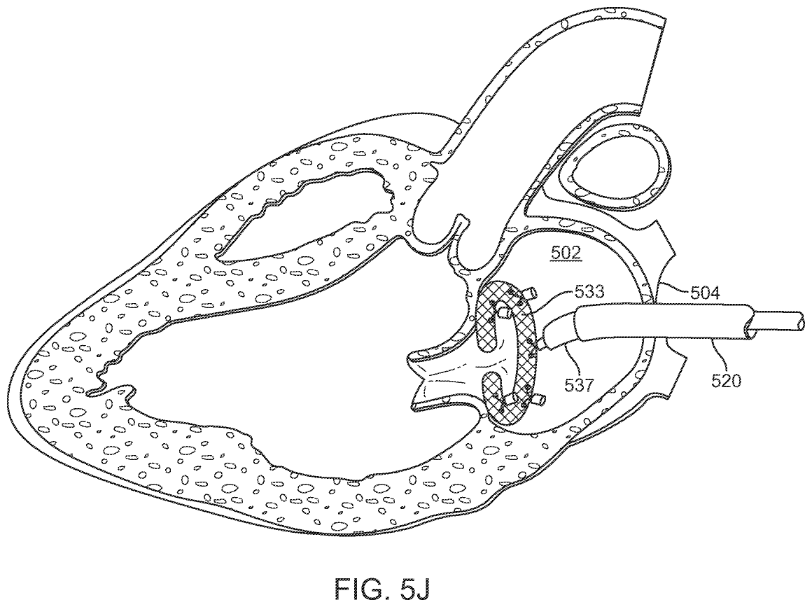

In some embodiments, percutaneous device 400 may comprise an access device configured to penetrate the atrial wall to provide an access channel into the left atrium. FIGS. 5A-5J are schematic diagrams illustrating the introduction of a percutaneous access device 520 into the left atrium of the heart.

FIG. 5A shows an example parasagittal cross-section of a heart 110. Also shown in FIG. 5A are left atrium 502 and mitral valve 506. A wall of left atrium 502 includes a portion called the roof or the dome 504.

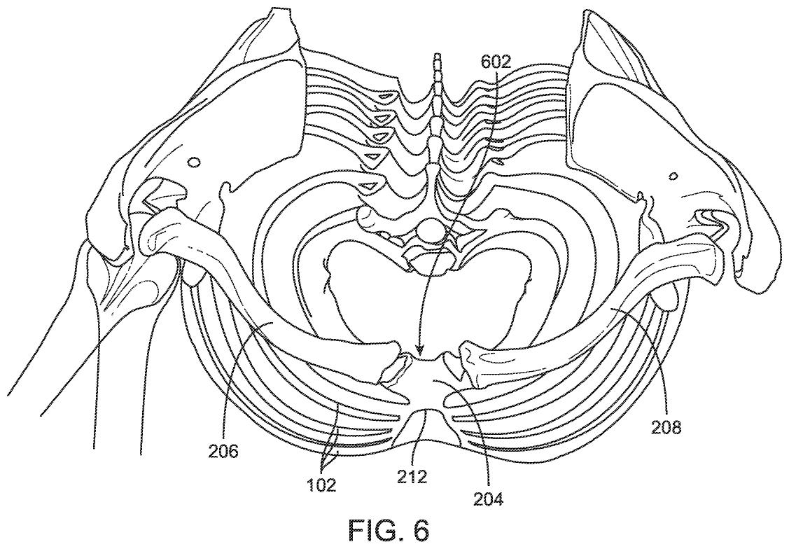

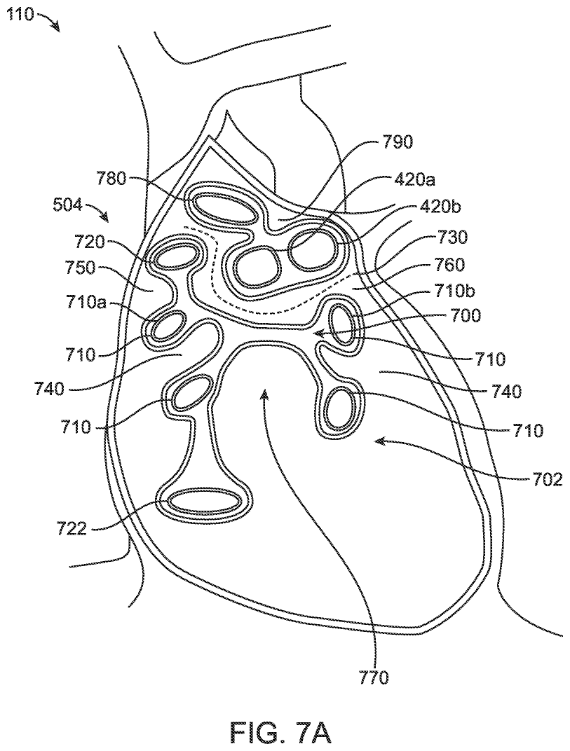

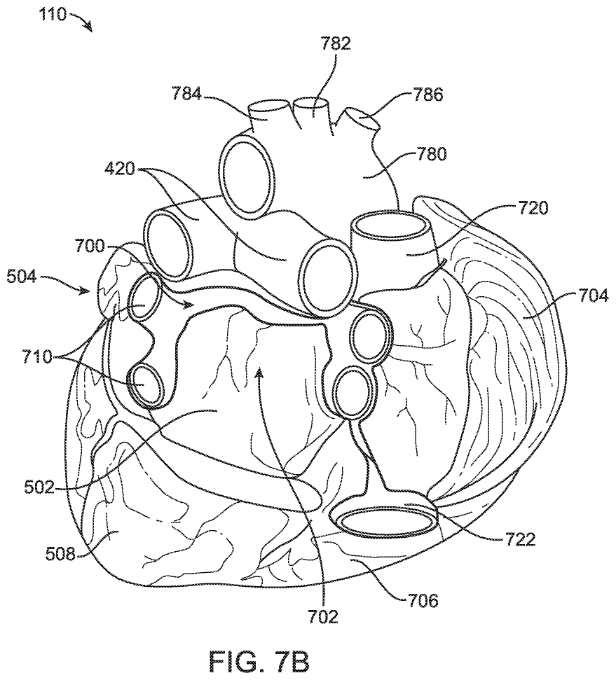

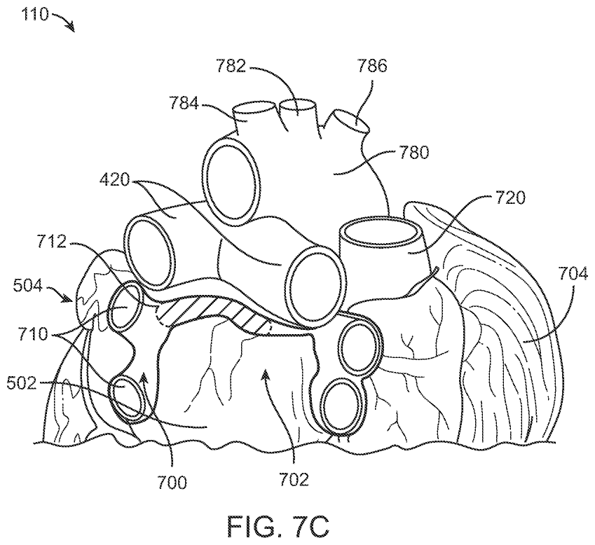

As shown in FIG. 5B, access device 520 is advanced until it is in close proximity or in contact with the wall of left atrium 502. Preferably, access device 520 is positioned to contact the roof or dome 504 of the left atrium in a location which lies outside the pericardium of the heart, as described below in connection with FIGS. 7A-7C.

In some embodiments, access device 520 includes a sensor 530 at its distal tip. Sensor 530 can be configured to determine whether the access device is in contact with the cardiac wall. The sensor may comprise a proximity sensor, capacitive sensor, contact sensor, infrared sensor, audio sensor, ultrasound transducer, or other known type of sensor.

The access device 520 may be configured to form a penetration (e.g. make an incision, puncture, or the like) at a target location in the wall of the heart to allow access into selected chambers of the heart. In specific embodiments the chamber is an atrium, more preferably the left atrium. In particularly preferred embodiments, an atrial penetration is made in the roof or dome 504 of the left atrium without penetrating the pericardium of the heart 110 or entering pericardial cavity or sac (referred to herein as an extrapericardial penetration, extrapericardial puncture, an extrapericardial incision). Such an extrapericardial penetration may avoid complications of conventional trans-pericardial surgical approaches such as unintentional injury to the heart wall and/or pericarditis. Additionally dome 504 of the left atrium 502 is relatively immobile, which may make it easier to form a penetration at that location during beating heart procedures in comparison to, for example, the left ventricle which is entered in trans-apical procedures. Further, by eliminating the need to penetrate or open the pericardium, the need for specialized techniques and instruments for entering the pericardium safely may be obviated.

In some embodiments, an atrial penetration may be formed while the heart is beating. In some embodiments, the atrial penetration may be formed while the heart is slowed. In some embodiments, the atrial penetration may be formed while the heart is stopped. In some embodiments, the atrial penetration may be formed when the heart is on cardiopulmonary bypass.

In stopped heart procedures, a patient may be placed on cardiopulmonary bypass without incisions in the chest by placing an endoaortic occlusion catheter into a femoral or iliac artery and advancing it into the ascending aorta, where a balloon may be expanded to occlude the aorta as will be known to one of ordinary skill in the art. A femoral venous cannula may be used to withdraw blood from the patient and deliver it to an external oxygenator and pump, from which blood may be returned to the patient via a femoral arterial cannula as will be known to one of ordinary skill in the art.

FIGS. 7A-7C show the extrapericardial location 700 outside pericardium 702 on the roof 504 (also referred to herein as the dome) of the left atrium 502 of the heart 110. FIGS. 7A-7B show diagrams of the infero-posterior surface of the heart 110, highlighting the dome 504 of the left atrium 502. An extrapericardial portion 700 of the roof 504 of the left atrium 502 may be located in a space between the four ostia of the pulmonary veins 710 the superior vena cava 720, and the inferior vena cava 722. In some cases, the extrapericardial portion 700 may be an elongated rectangular, arch-shaped, or undulating space on the left atrial roof 504 approximately 1 cm to about 6 cm in length and 0.5 cm to about 3 cm in width, extending generally between the left superior pulmonary vein 710a and the right superior pulmonary vein 710b. An extrapericardial portion 700 of the roof 504 of the left atrium 502 may be bounded by the transverse sinus 730, the pulmonary venous recesses 740, the post caval recess 750, the left pulmonic recess 760, and the oblique sinus 770. An extrapericardial portion 700 of the roof 504 of the left atrium 502 may be located in a space between the ostia of the aortic root 780, the right pulmonary artery 420a, and the left pulmonary artery 420b. An extrapericardial portion 700 the roof 504 of the left atrium 502 may be bounded by the transverse sinus 730 and the superior pericardial recess 790. An extrapericardial portion 700 of the roof 504 of the left atrium 502 may be located in a space between the ostia of the four pulmonary veins 710, the intra-pericardial portion of the posterior wall of the left atrium 504, and the pulmonary arteries 420. The extrapericardial portion 700 of the roof 504 of the left atrium 502 may enlarge as the left atrium 504 enlarges.

FIG. 7C shows a diagram of the heart 110, highlighting an exemplary target extrapericardial location 712 on the roof 504 of the left atrium 502. The target extrapericardial location 712 may be a target location through which the percutaneous device or other instrument(s) may access an interior portion of the heart 110 (e.g. via a puncture, an incision, or other opening 512 therein). The target location 712 (e.g. target location of an opening or incision or puncture in the cardiac wall) may be a space on the left atrial wall in a space between at least two pulmonary vein ostia 710. The target location 712 may be in the left atrial wall in a space between four pulmonary vein ostia 710. The target location 712 may be accessed by the percutaneous device as described herein without accessing, puncturing, or penetrating major vessels of the heart (e.g. the left carotid artery 782, the left subclavian artery 784, or the brachiocephalic trunk 786). The target location 712 may be accessed by the percutaneous device as described herein without access in the right atrium 704, the right ventricle 706, or the left ventricle 508 as described herein.

Referring now to FIG. 5C, in some embodiments, access device 520 includes one or more components (e.g., one or more needles, blades, or other penetration devices) for making a puncture or penetration in the wall of left atrium 502. In one embodiment, access device 520 is configured to be introduced into the left atrium using an access technique similar to the Seldinger technique. With access device 520 positioned in contact with or in close proximity to the left atrial dome, a hollow needle 521 is advanced from a lumen within access device 520 to penetrate the left atrial wall and enter the left atrium. A wire 523 is then advanced from within needle 521 into the left atrium.

As shown in FIG. 5D, after the hollow needle 521 has been removed, a tapered-tip dilator 525 may then be advanced from within access device 520 over guidewire 523 to further widen the penetration through the left atrial wall. Finally, as shown in FIG. 5E, access device 520 is inserted through left atrial wall over dilator 525. Dilator 525 may then be removed from access device 520.

It will be understood that in some embodiments dilator 525 may not be necessary. For example, access device 520 may be configured with a rounded or tapered tip to facilitate introduction directly over hollow needle 521 and/or wire 523.

Due to the relatively low left atrial pressure and the tight fit of access device 520 in the penetration in the left atrial wall, hemostasis may in some cases be adequate without taking other steps to seal the penetration around access device 520. In other situations, it may be desirable to enhance such sealing. Various means may be used to provide hemostatic sealing around access device 520. In one embodiment, a purse-string suture may be placed in the left atrial wall around the penetration through which access device 520 extends. Such purse-string suture is preferably placed prior to introduction of access device 520. One example of an endoscopic device suitable for placement of such a purse-string suture is disclosed in commonly assigned PCT Application Serial No. PCT/US2019/012538, filed Jan. 7, 2019, (Attorney Docket No. 54513-704.601), the disclosure of which is incorporated herein by reference.

Alternatively, other types of endoscopic devices may be used to create a hemostatic seal around the atrial penetration prior to introduction of access device 520. For example, an endoscope may be inserted through introducer sheath 210 and advanced to the left atrial dome in the manner described above. A suturing device may then be inserted through the working channel of the endoscope to place one or more sutures in the left atrial wall adjacent to or around the site at which access device 520 is to be inserted. The suture ends may be withdrawn from the mediastinum through introducer sheath 210 and, following introduction of access device 520 into the left atrium, cinched in order to tightly seal the atrial penetration around access device 520. Following removal of atrial access device 520, such sutures may be tied to close the atrial penetration.

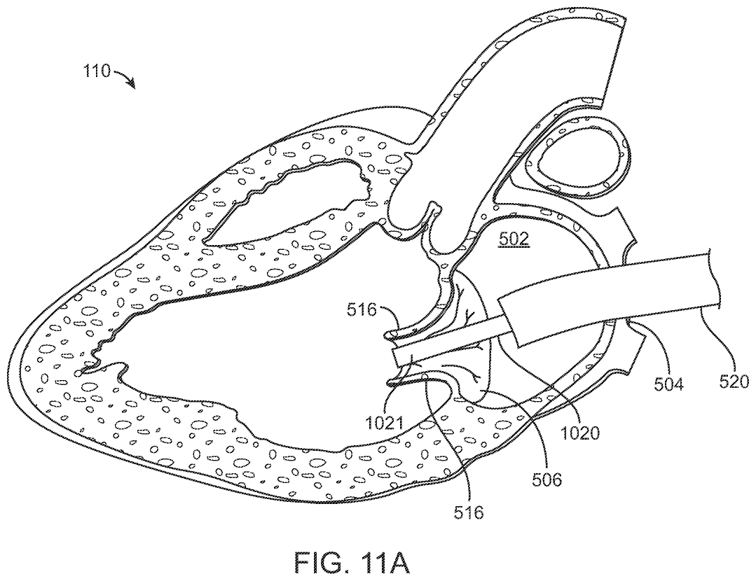

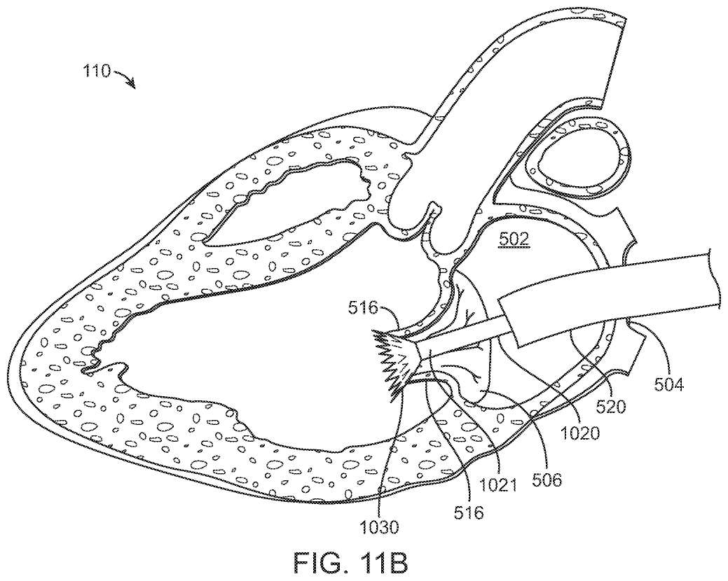

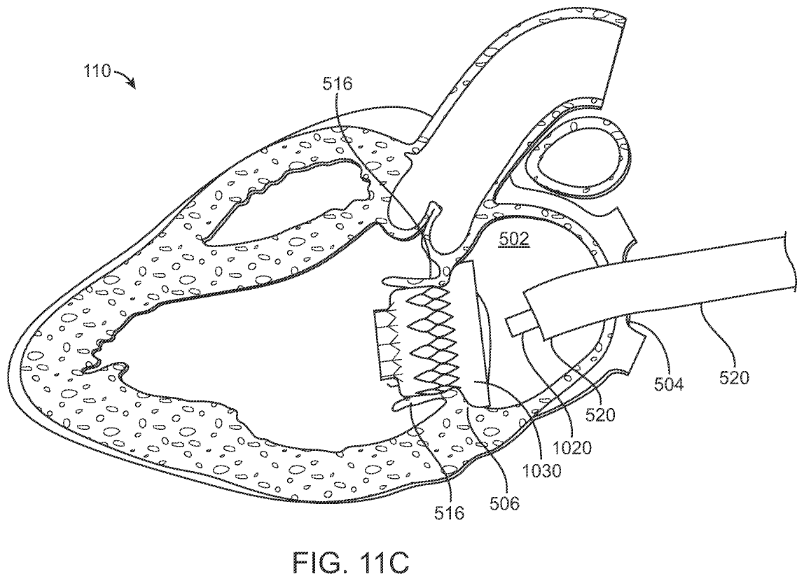

In other embodiments, access device 520 may itself include means for sealing the atrial penetration to establish hemostasis. For example, access device 520 may include an inflatable balloon or mechanically expandable flange around its periphery in a distal region thereof configured to engage the interior atrial wall around the penetration, as disclosed in the aforementioned PCT Application No. PCT/US18/42171, which has been incorporated herein by reference. Such patent application also discloses various mechanisms for deploying needles and sutures from a left atrial access device for purposes of both sealing and closing an atrial penetration, any of which may be incorporated into access device 520. Other access devices incorporating penetration closure devices are described below in connection with FIGS. 11A-11H.

The access device 520 may, for example, have an outer diameter of about 3 mm to about 20 mm, usually about 3 mm to about 15 mm, and more preferably about 3 mm to about 10 mm. The access device 520 may have a length of about 5 cm to about 60 cm from the proximal end to the distal end, usually about 10 cm to about 40 cm, and preferably about 15 cm to about 30 cm.

The access device 520 may comprise a channel 540 extending therethrough from a proximal end of the access device 520 to a distal end of the access device 520. The channel may be defined by an inner wall of the access device 520 having an inner diameter. In some embodiments, the access device 520 may comprise a cannula, sheath, or tube. The channel may have a diameter of about 1 mm to about 12 mm, usually about 1 mm to about 10 mm, or more preferably about 2 mm to about 8 mm.

In some embodiments, channel 540 of the access device 520 may be configured to allow one or more additional members to be slidably and/or removably disposed therein. The access device 520 may, for example, be configured to allow one or more of a penetration device, a closure device, a sealing device, a procedural device, a visualization device, a prosthesis delivery device, and/or a suturing device to access the left atrium 504 as described herein. In some embodiments, the access device may include an internal sealing element to inhibit blood loss through the channel therein. The internal sealing element may comprise a hemostatic valve disposed in the channel and configured to inhibit blood loss therethrough. The hemostatic valve may comprise, for example, a duck bill valve, a diaphragm valve, a touhy-borst valve, or a three leaflet valve.

Access device 520 may optionally comprise an anchoring element (not shown) coupled to a proximal portion thereof configured to be positioned either externally or in the mediastinum near the suprasternal access site. The anchoring element may be configured to inhibit movement of access device 520 relative to the patient to prevent inadvertent removal of the percutaneous device from the heart through the atrial penetration, or inadvertent advancement toward or within the heart beyond a desired distance. The anchoring element may comprise a ring, flange, laterally-extending handles or wing-like elements, or other suitable structure on the proximal portion of the access device configured to engage the patient's body, surgical drapes, or other material adjacent the suprasternal opening. Alternatively, the anchoring element may comprise a mechanical arm attachable to access device 520 and coupled to a stationary structure such as the operating table.

Access device 520 may optionally comprise a retention element coupled to a distal portion thereof. The retention element may be configured to prevent inadvertent removal of the access device through the atrial penetration. In some embodiments, the retention element may comprise a flange, a ring, an expandable wire basket, deployable wing-like elements, or a balloon. The retention element may have an undeployed configuration to aid in advancement of the device to the heart and through the atrial wall, and a deployed configuration configured to resist inadvertent removal of the elongate member from a cardiac wall of the patient.

Referring to FIGS. 5E-5F, in one embodiment access device 520 comprises a steerable sheath which can be controlled from its proximal end to place its distal tip 527 into a desired orientation or shape. For example, access device 520 may include one or more steering wires (not shown) extending through axial lumens in the device and secured in a distal region of distal tip 527. Using a mechanism at the proximal end of access device 520, the steering wires can be tensioned in order to bend distal tip 527 in one or more directions and at various radii of curvature. This allows access device 520 to be steered along a curved or angled path to the mitral valve or other location of interest. Further, such steerability allows distal tip 527 to be placed in a desired orientation and angle relative to the valve or other target structure.

Once access device 520 is positioned at the desired location in the left atrium, a procedural catheter 524 can be inserted through a channel of access device 520 into the left atrium in order to perform a procedure within the heart. The procedure may comprise at least one of mitral valve replacement, mitral valve repair, mitral annuloplasty, chordal repair, chordal replacement, leaflet resection, mitral replacement, leaflet coaptation, papillary repair, or papillary coaptation. The procedure may alternatively comprise at least one of atrial appendage closure, atrial ablation, pulmonary vein ablation, septal defect closure, aortic valve repair, aortic valve replacement, tricuspid valve repair, tricuspid valve replacement, implantable cardiac defibrillator (ICD) implantation, pacemaker implantation, or placement of leads for ICD's or pacemakers, myocardial biopsy, or septectomy.

Similar to access device 520, procedural catheter 524 will be adapted for visualization using fluoroscopy, TEE, transthoracic echocardiography or other indirect visualization technique. Procedural catheter 524 may be composed of radiopaque or echogenic materials, and/or include radiopaque or echogenic markers at one or more locations along its length. Such markers can be viewed in relation to the position of markers on access device 520, on guidewire 523, or on other devices such as a tracheal tube or an esophageal probe to assist in positioning. Procedural catheter 524 may further have lumens, chambers, or inflatable elements that can be filled with radiopaque fluid. Alternatively, procedural catheter 524 may be configured to inject radiopaque dye into the mediastinum or heart during the procedure to facilitate visualization of its location.

While access device 520 is preferably steerable as previously described, alternatively or additionally procedural catheter 524 may have a steerable distal portion. Providing steerability in both access device 520 and procedural catheter 524 allows highly precise multi-axis positioning of a distal end of procedural catheter 524 to facilitate various interventional procedures. Various known mechanisms may be used to enable such steerability, such as steering wires extending slidably through one or more lumens in procedural catheter 524 and secured at its distal tip. Such wires can be tensioned to bend procedural catheter 524 around one or more axes using a control mechanism at the proximal end of the catheter.

In an exemplary embodiment, the procedure is mitral annuloplasty, as illustrated in FIGS. 5F-5J. FIG. 5F illustrates procedural catheter 524 in the form of a suture anchor placement device. Access device 520 has been steered into the desired position and orientation, and procedural catheter 524 is advanced distally from access device 520 to engage the tissue of the mitral annulus. A suture anchor 529 is held at the distal end of procedural catheter 524 which is configured to deploy suture anchor 529 into the mitral annulus. One or more sutures 531 are coupled to anchor 529 and extend proximally out of the patient through access device 520.

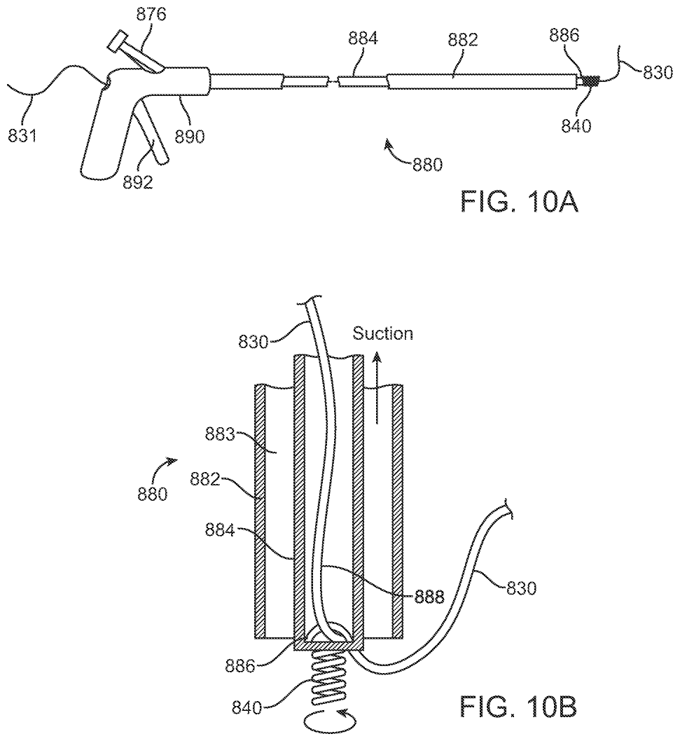

Suture anchor 529 may have a variety of configurations. In an exemplary embodiment, suture anchor 529 comprises a helical coil adapted to be rotationally driven into the annulus. One embodiment of a device for delivering such a helical suture anchor is described in connection with FIGS. 10A-10B, below. In this embodiment, procedural catheter 524 includes a rotational drive mechanism to rotationally drive suture anchor 529 into the tissue, then release suture anchor 529 from its distal end. Procedural catheter 524 may then be removed from access device 520, while the proximal extremities of suture 531 remain outside the patient's chest.

Under fluoroscopic visualization and/or TEE, access device 520 is then steered to another location along the mitral annulus, and procedural catheter 524, loaded with another suture anchor 529B, may be inserted through access device 520, as shown in FIG. 5G. Suture anchor 529B is rotationally driven into the second location on the annulus, and procedural catheter 524 is again withdrawn from access device 520, leaving a second pair of suture extremities 531B extending out of the patient's chest.

Referring to FIG. 5H, after a plurality of suture anchors 529 are implanted in the annulus, sutures 531 can be placed through an annuloplasty band or ring 533 outside the patient's chest. The annuloplasty band 533 is releasably coupled to a delivery catheter 535 adapted to insert annuloplasty band 533 through the channel of access device 520. Annulopasty band 533 slides along sutures 531 as it is inserted. Annuloplasty band 533 is advanced into the left atrium and positioned against the mitral annulus MA, as shown in FIGS. 5H-5I, with sutures 531 extending out of the chest through access device 520.

Sutures 531 are then tightened and secured using knots or other devices. In one embodiment, knots are tied in each pair of suture extremities 531 outside the patient's chest, and an elongated flexible knot pushing device (not shown) is used to push each knot through access device 520 to engage annuloplasty band 533. The suture ends are then trimmed using a trimming catheter (not shown). Alternatively, a catheter 537 adapted to deploy crimpable fasteners similar to the Cor-Knot™ device may be used to secure and trim the sutures, leaving the annuloplasty ring 533 securely anchored in place, as shown in FIG. 5J.

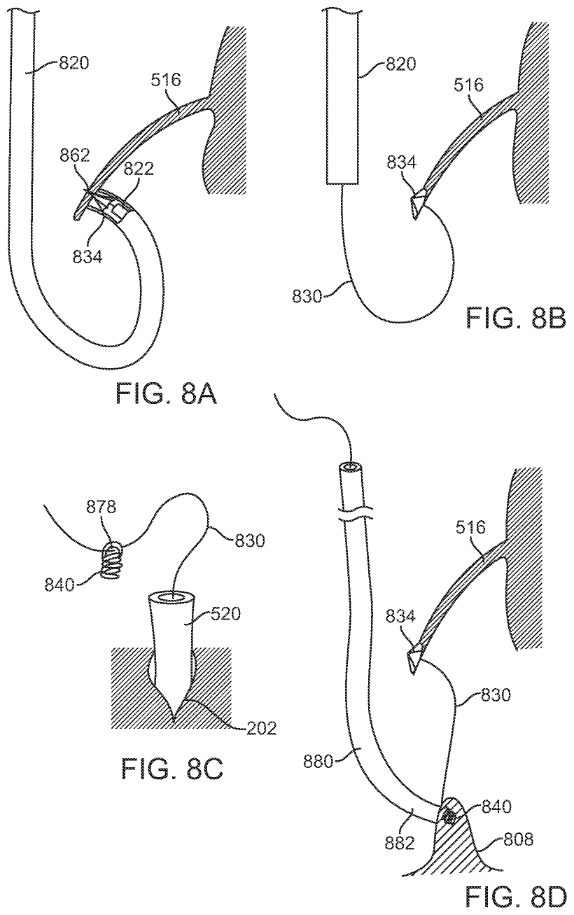

In another embodiment, procedural catheter 524 comprises a chordal repair catheter 820 configured to perform replacement of chordae tendineae, as illustrated in FIGS. 8A-8G. The chordal replacement procedure is preferably performed while the heart is beating. A chordal repair catheter 820 may be advanced into the left atrium through the channel of the access device 520 (not shown in FIGS. 8A-8G). Preferably, access device 520 will be steerable to facilitate accurate positioning of chordal repair catheter in the heart. Alternatively, chordal repair catheter 820 may be introduced directly into the left atrium without the use of access device 520. In such embodiments, chordal repair catheter 820 will preferably be steerable. The chordal repair catheter 820 may, for example, comprise a distal end effector 822 configured to couple one or more replacement chords 830 to at least one of a mitral valve leaflet 516 of the patient and a papillary muscle 808 of the patient to form one or more artificial chordae tendineae therebetween.

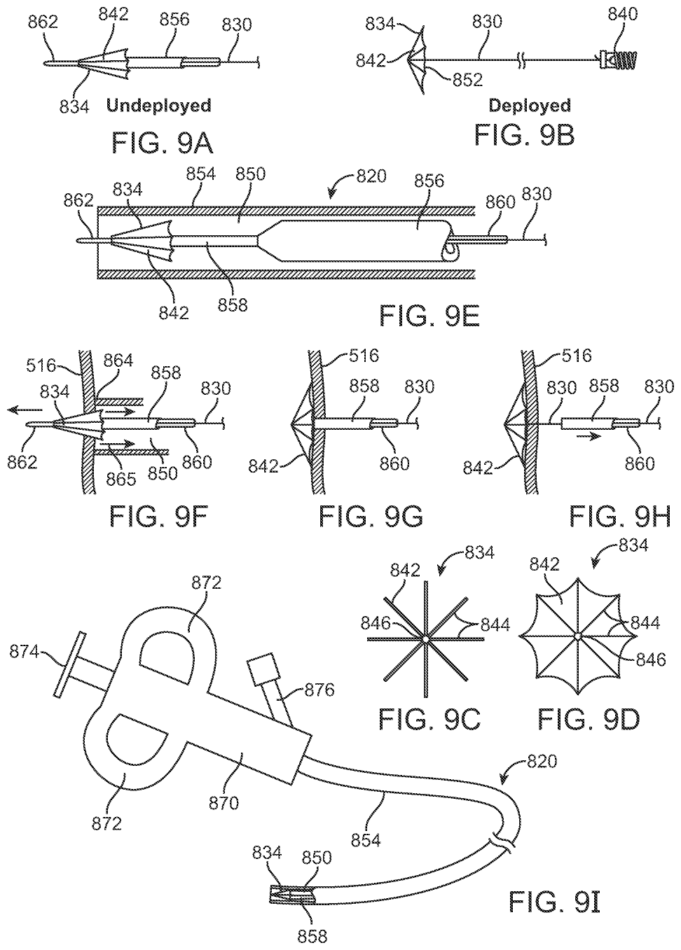

In an exemplary embodiment, illustrated in FIGS. 9A-9I, the replacement chords 830 may comprise a flexible strand, suture, wire, or chord with a leaflet anchor 834 on a free end thereof. In one embodiment, replacement chord 830 comprises a polytetrafluoroethylene (PTFE) suture. The chordal repair catheter 820 may be configured to releasably hold the leaflet anchor 834 with the distal end effector 822 and secure it to a mitral leaflet 516. As shown in FIG. 9B, the opposing end of replacement chord 830 is configured for coupling to a papillary anchor 840, which may comprise, for example a helical coil anchor similar to that described above in connection with FIGS. 5G-J.

Leaflet anchor 834 comprises a radially collapsible and expandable retainer 842 that in exemplary embodiments has a plurality of radial spokes 844, coupled to a cylindrical central hub 846, as shown in FIGS. 9C-9D. Optionally, retainer 842 may include a membrane 848 extending over spokes 844 comprising a thin sheet of a flexible, biocompatible material such as Dacron or ePTFE which may be on either the inside/proximal surface or outside/distal surface of retainer 842, which may reduce trauma and encourage tissue in-growth on the leaflet 516. Retainer 842 has a collapsed configuration, shown in FIG. 9A, suitable for positioning within a lumen 850 of chordal repair catheter 820, as shown in FIG. 9E. In the collapsed configuration, retainer 842 is also adapted to be passed through leaflet 516 as further described below. Retainer 842, including spokes 844, are resiliently biased into an expanded configuration shown in FIG. 9B in which spokes 844 point radially outward at an angle of about 60-90° relative to the longitudinal axis. In this configuration, an inner/proximal surface 852 is configured for atraumatic engagement with surface of leaflet 516 and to resist pulling through or tearing the leaflet when the replacement chord is under tension. Spokes 844 are composed of a suitably resilient, shape memory material with suitable stiffness to resist deformation and retain the replacement chord in the leaflet, e.g. a high-elasticity material such as stainless steel or Nitinol.

Referring to FIG. 9E, leaflet anchor 834 is releasably retained in lumen 850 of an outer shaft 854 of chordal repair catheter 820. A tubular pusher 856 is slidably disposed within lumen 850 and has a distal tip 858 which engages a proximal side of hub 846. A tubular inner shaft 860 is slidably disposed within pusher 856 and has a distal needle 862 extending through hub 846. A replacement chord 830, e.g. suture, extends through pusher 856 alongside inner shaft 860 and is attached at its distal end to hub 846.

FIG. 9I illustrates the complete chordal replacement catheter 820. At its proximal end, outer shaft 854 is attached to a handle body 870 having a pair of finger grips 872. A plunger 874 is slidably coupled to handle body 870 and configured to be actuated by the user's thumb. Plunger 874 is coupled to pusher 856 and inner shaft 860, such that by pushing the plunger 874 axially needle 862 and leaflet anchor 834 are advanced distally relative to outer shaft 854. Optionally, separate actuators may be provided on handle body 870 to allow independent actuation of pusher 856 and inner shaft 860. A suction port 876 on handle body 870 is fluidly coupled to lumen 850 to allow application of suction thereto.

Chordal replacement catheter 820 is preferably steerable (in addition to access device 520) to facilitate steering the distal tip thereof into engagement with leaflet 516. Known catheter steering mechanisms may be used for this purpose. In preferred embodiments, chordal replacement catheter is configured to be positioned from a suprasternal access site into the left atrium, through the mitral valve, around the edge of a mitral leaflet, and into engagement with a downstream or ventricular surface of the leaflet, as shown in FIG. 8A.

FIGS. 9F-9H illustrate the placement of leaflet anchor 834 in leaflet 516. A distal end 864 of outer shaft 854 may be placed in engagement with leaflet 516 and suction may be applied through lumen 850 as shown by arrows 865. Needle 862 may be advanced distally from outer shaft 854 and pusher 858 is used to advance leaflet anchor 834 together with needle 862 to penetrate leaflet 516, as shown in FIGS. 8A and 9F. Upon passage completely through leaflet 516 retainer 842 expands to the expanded configuration shown in FIG. 9G. Needle 862 (via inner shaft 860), along with pusher 858, may then be retracted from leaflet anchor 834, and suction discontinued to allow chordal repair catheter 820 to be withdrawn from leaflet 516, with replacement chord 830 remaining coupled to the leaflet via leaflet anchor 834. Replacement chord 830 is allowed to slide within pusher 856 as the catheter is withdrawn, thus leaving the arrangement shown in FIG. 8B.

Referring to FIG. 8C, leaflet repair catheter 820 may be withdrawn from the patient's chest and decoupled from replacement chord 830, leaving the replacement chord 830 extending out of the suprasternal penetration 202 via access device 520. A free end of replacement chord 830 is then slidably coupled to papillary anchor 840, e.g. by passing the free end through an eyelet 878 on papillary anchor 840.

As shown in FIG. 8D, an anchor delivery catheter 880 may be used to deliver papillary anchor 840 into the heart and implant it in the papillary muscle 808. Anchor delivery catheter 880 is shown in greater detail in FIGS. 10A-10B. Anchor delivery catheter 880 has an outer shaft 882, which is preferably steerable using a known catheter steering mechanism, as, for example, described above in connection with access device 520 and procedural catheter 524. An inner drive shaft 884 is disposed within a lumen 883 of outer shaft 882 and is rotatable around a longitudinal axis thereof. A distal end 886 of drive shaft 884 is releasably coupled to papillary anchor 840 while allowing replacement chord 830 to remain slidably coupled thereto. Papillary anchor 840 may, for example, be frictionally retained within inner lumen 888 in drive shaft 884, with a slot or other feature preventing rotational slippage therein.

A handle 890 is coupled to the proximal end of outer shaft 882, as shown in FIG. 10A. An actuator 892 is movably coupled to handle 890 and is connected to drive shaft 884 via rack and pinion gear system or other suitable mechanism (not shown) such that moving actuator 892 causes drive shaft 884 to rotate within outer shaft 882, thus driving papillary anchor 840 in a rotational motion. A suction port 896 on handle 890 is fluidly coupled to lumen 883 to allow suction to be applied therethrough. Free end 831 of replacement chord 830 passes through inner lumen 888 and through an exit port 898 in handle 890.

Referring again to FIG. 8D, papillary muscle 808 may be engaged with the distal end of outer shaft 882 and suction applied therethrough to securely adhere catheter 880 to the papillary muscle. Actuator 892 (not shown in FIG. 8D) may then be actuated to drive papillary anchor 840 rotationally into papillary muscle 808. Suction may then be discontinued and anchor delivery catheter 880 withdrawn from the patient, with a free end of replacement chord 830 remaining outside the chest.

Replacement chord 830 is then adjusted and secured. In an exemplary embodiment, shown in FIG. 8E, free end of replacement chord 830 is coupled to a crimping device 900 carried by a crimping catheter 902. Crimping device 900 may have various embodiments, for example, a device similar to a Cor-Knot™ fastener. Using crimping catheter 900, crimping device 900 is slidably advanced along replacement chord 830 up to papillary anchor 840, as shown in FIG. 8F.

Before finally securing replacement chord 830 it is adjusted in length and tension to achieve optimal valve function and minimize mitral regurgitation. While observing the mitral valve using TEE and/or fluoroscopy, replacement chord 830 may be tensioned by the operator until regurgitation is minimized. The operator then actuates crimping catheter 902 to simultaneously crimp crimping device 900 and trim off the free end of replacement chord 930. Crimping catheter 902 is then removed from the patient, leaving the completed repair shown in FIG. 8G.

The foregoing process may be repeated to place multiple replacement chords 830 as needed.

FIGS. 11A-11C illustrate an exemplary percutaneous system and method for mitral valve replacement (for clarity the native chordae tendineae are not shown). The mitral valve replacement procedure may be performed while the heart 110 is beating. In this embodiment procedure catheter 524 comprises a valve delivery catheter 1020. Valve delivery catheter 1020 may be advanced into the left atrium 502 of the heart 110 through the channel of access device 520 and used to perform a mitral valve replacement procedure. Alternatively, valve delivery catheter 1020 may be introduced from a suprasternal access site into the left atrium directly, without the use of access device 520. In preferred embodiments, either or both access device 520 and/or valve delivery catheter 1020 are steerable to facilitate positioning the valve prosthesis properly relative to the native valve.

Valve delivery catheter 1020 may comprise a sheath 1021 or capsule in a distal region thereof configured to hold a prosthetic mitral valve 1030 (shown in FIG. 11B). The prosthetic mitral valve 1030 may, for example, be a stented mitral valve, e.g. any suitable stented prosthetic valve which can be collapsed to a delivery diameter less than about 20 mm, preferably less than about 15 mm, and more preferably less than about 10 mm, which is suitable for placement using a left atrial approach, and which can be expanded to a diameter large enough to engage the mitral annulus. The prosthetic mitral valve 1030 may be contained in the sheath 1021 in a collapsed configuration while the valve delivery catheter 1020 is advanced into the internal chamber of the heart 110 as shown in FIG. 11A. The valve delivery catheter 1020 may, for example, be advanced from the penetration in atrial dome 504 to a location between the mitral leaflets 516, for example about 4 cm to about 8 cm into the left atrium 502 depending on the size of the left atrium 502 of the patient.

Once the distal end of the valve delivery catheter 1020 has been advanced into the desired position and/or orientation, the sheath may be retracted and the prosthetic mitral valve 1030 may be released (as shown in FIG. 11B) and expanded into an undeformed shape (as shown in FIG. 11C) inside the native mitral valve 506. The prosthetic mitral valve 1030 may be resiliently deformable into the collapsed configuration within delivery catheter 1020 and configured to resiliently return to its undeformed shape upon deployment from the catheter. In some embodiments, the native mitral valve leaflets 516 may remain in place prior to and after implantation of the prosthetic mitral valve 1030. Proper positioning and orientation of the prosthetic mitral valve 1030 may be visualized before, during, and/or after implantation using any of the visualization techniques described herein, for example, fluoroscopy, TEE, and/or transthoracic echocardiography.

After accessing the internal chamber of the heart and/or preforming one or more cardiac procedure therein, the distal portion of the percutaneous device may be removed from the heart and the atrial penetration may then be closed. Any of the percutaneous devices or systems described herein may optionally comprise a closure device.

In some embodiments, the atrial penetration may be closed by cinching a purse string suture placed circumferentially around the atrial penetration as described herein.

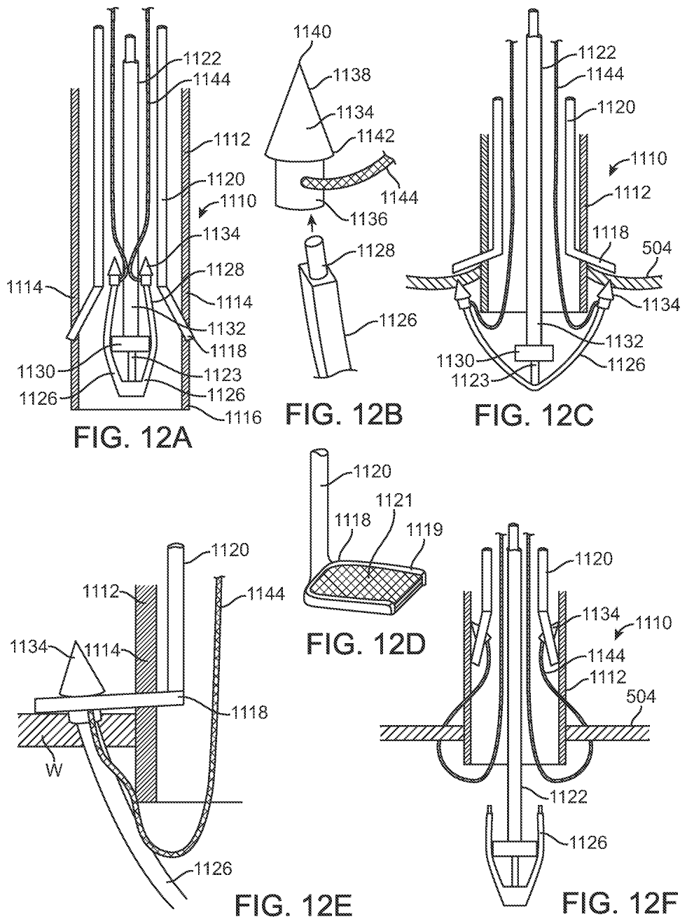

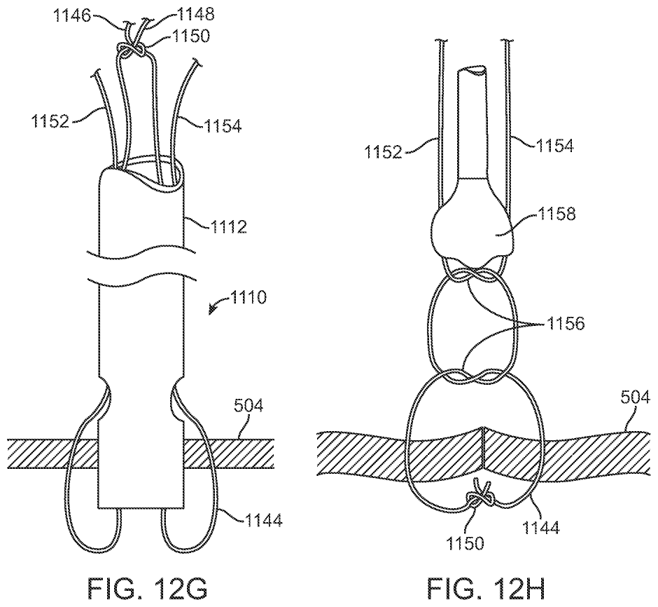

In some embodiments, the atrial penetration may be closed with the aid of one or more closure device (also referred to herein as a suturing device) as described herein. An exemplary embodiment of such a closure device is illustrated in FIGS. 12A-12H. As shown in FIG. 12A, closure device 1110 comprises a tubular outer shaft 1112 having a pair of exit ports 1114 near its distal end 1116. A pair of catchers 1118 attached to catcher shafts 1120 are aligned with exit ports 1114 and axially movable within outer shaft 1112. As shown in FIG. 12D, each catcher 1118 has an outer frame 1119 supporting a porous mesh 1121 configured to allow penetration by a needle, as further described below. Frame 1119 is biased into a transverse orientation relative to catcher shaft 1120, as shown in FIG. 11D.

A needle assembly 1122 is disposed in outer shaft 1112 between catchers 1118 and is axially movable relative thereto. Needle assembly 1122 has an inner shaft 1123 having a distal end 1124 to which a pair of needle arms 1126 is coupled. Needle arms 1126 have arm ends 1128 pointing in a proximal direction relative to outer shaft 1112 and are deflectable from an inward position in which they are contained within outer shaft 1112, shown in FIG. 12A, to an outward position in which arm ends 1128 extend outside outer shaft 1112, as shown in FIG. 12C. The needle arms 1126 are resiliently biased to the inward position and can be deflected outwardly by advancing a cam member 1130 attached to an actuator shaft 1132, which is slidably disposed over inner shaft 1123.

As shown in FIG. 12B, a needle tip 1134 is releasably coupled to each arm end 1128 of needle arms 1126. Needle tips 1134 have a socket portion 1136 configured to slide over arm end 1128 and a tapered head 1138 having a sharp distal point 1140 configured to penetrate heart tissue and a widened proximal flange 1142. Socket portion 1136 may form a frictional engagement with arm end 1128, or a detent or other feature may be included to provide more positive engagement. A pair of sutures 1144 are respectively attached to needle tips 1134 and extend through outer shaft 1112 to its proximal end.

Closure device 1110 may be introduced through access device 520 into the left atrium, or directly introduced through an atrial penetration. If introduced through access device 520, once distal end 1116 is positioned in the left atrium, access device 520 may be retracted from the atrial penetration, leaving only closure device 1110 therein. As shown in FIG. 12C, needle assembly 1122 may be advanced relative to outer shaft 1112 until needle arms 1126 are distal to distal end 1116. Actuator shaft 1132 may then be advanced distally relative to inner shaft 1123 such that cam member 1130 urges needle arms 1126 to the outward position. Catcher shafts 1120 are moved distally within outer shaft 1112 such that catchers 1118 move outwardly through exit ports 1114 and extend over an outer surface of atrial roof 504.

Needle assembly 1122 is then retracted proximally relative to outer shaft 1112, driving needle tips 1134 through the atrial wall and through mesh 1121 of catchers 1118, as shown in FIG. 12E. It may be seen that sutures 1144 extend from outer shaft 1112 through the atrial wall W.

With needle tips 1134 passed completely through mesh 1121, needle assembly 1122 is moved distally relative to outer shaft 1112. This decouples needle arms 1126 from needle tips 1134, which are retained in mesh 1121 by flanges 1142. As shown in FIG. 12F, catcher shafts 1120 are retracted proximally within outer shaft 1112, drawing needle tips 1134 and sutures 1144 proximally through the outer shaft so that both ends of each suture 1144 are disposed outside the patient's chest, thus creating a loop through each opposing tissue flap of the atrial penetration.

In an alternative embodiment, not shown, an additional tubular external shaft is slidably disposed over outer shaft 1112, and catchers 1118 are coupled to the distal end of this second external shaft. In this way, catchers 1118 remain outside outer shaft 1112 while they are withdrawn from the mediastinum using the external shaft, leaving one extremity of each suture 1144 outside outer shaft 1112 while the other extremity is within outer shaft 1112.

As shown in FIG. 12G, a first pair of ends 1146, 1148 of sutures 1144 may be interconnected outside the patient, by e.g. a knot 1150. Second pair of ends 1152, 1154 may then be pulled to draw knot 1150 distally through outer shaft 1112 into the left atrium. Closure device 1110 may then be withdrawn from the atrial penetration, allowing sutures 1144 to slide through exit ports 1114 until second ends 1152, 1154 are removed from the device. Second ends 1152, 1154 may then be tightened and secured, e.g. by tying knots 1156 and advancing them to the atrial dome 504 using an endoscopic knot pushing device 1158. Alternatively, a crimped fastener such as a Cor-Knot device may be used to secure the sutures.

It will be appreciated that, while closure device 1110 may be placed through access device 520 to close the atrial penetration following a procedure, closure device 1110 may alternatively be used in place of access device 520 to provide a channel through which a procedural catheter 524 or other device may be inserted into the heart. In such embodiments, needle assembly 1122 and catchers 1118 are initially removed from outer shaft 1112 and replaced with a needle, obturator and guidewire assembly similar to that described above in connection with FIGS. 5A-5E. The needle, obturator, and guidewire assembly are used to create a penetration through the atrial wall through which outer shaft 1112 is inserted. The needle, obturator, and guidewire assembly are then removed and replaced with needle assembly 1122 and catchers 1118, which may be used to pass sutures 1144 through the atrial wall on opposing sides of the penetration therein. The sutures may be tensioned during the procedure in order to seal the atrial wall against outer shaft 1112 and prevent loss of blood. Procedural catheter 524 and/or other devices may be passed through outer shaft 1112 to perform a procedure in the heart. In some embodiments, outer shaft 1112 may include a hemostasis valve therein which permits passage of devices therethrough while inhibiting loss of blood. Following the procedure, outer shaft 1112 may be removed from the penetration and sutures 1144 secured in the manner described above.

In some embodiments, access device 520 and/or procedural catheter 524 may be coupled to a robotic manipulator disposed outside a chest of the patient. The robotic manipulator may, for example, comprise a robotic arm positioned above the suprasternal opening 202. Alternatively, a portion of the robotic manipulator may be disposed inside a chest of the patient. The robotic manipulator may be controlled by an operator working at a remote-control console. Procedural catheters 524 having procedure-specific end-effectors maybe advanced through the channel of the access device 520 and manipulated by the robotic manipulator to carry out the desired procedure in the internal chamber of the heart 110.

In some embodiments, a percutaneous device kit may comprise one or more devices described herein disposed within a sealed sterile package. The kit may comprise an access device 520 and a procedure catheter 524 in a sealed sterile package. The kit may further comprise any of the devices or elements described herein, or any of combination of the devices or elements described herein.

The terminology used in the description of the various described implementations herein is for the purpose of describing particular implementations only and is not intended to be limiting. As used in the description of the various described implementations and the appended claims, the singular forms “a”, “an” and “the” are intended to include the plural forms as well, unless the context clearly indicates otherwise. It will also be understood that the term “and/or” as used herein refers to and encompasses any and all possible combinations of one or more of the associated listed items. It will be further understood that the terms “includes,” “including,” “comprises,” and/or “comprising,” when used in this specification, specify the presence of stated features, integers, steps, operations, elements, and/or components, but do not preclude the presence or addition of one or more other features, integers, steps, operations, elements, components, and/or groups thereof.

The foregoing description, for purpose of explanation, has been described with reference to specific implementations. However, the illustrative discussions above are not intended to be exhaustive or to limit the scope of the claims to the precise forms disclosed. Many modifications and variations are possible in view of the above teachings. The implementations were chosen in order to best explain the principles underlying the claims and their practical applications, to thereby enable others skilled in the art to best use the implementations with various modifications as are suited to the particular uses contemplated.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

What is claimed is:

1. A method of performing an interventional procedure in a beating heart of a patient, the method comprising:
introducing a procedural catheter through a suprasternal penetration at a suprasternal access site into a mediastinum of the patient;
advancing the procedural catheter through the mediastinum to an atrial dome of the heart;
inserting the procedural catheter into a left atrium of the heart through a puncture in the atrial dome while the heart is beating; and
performing an interventional procedure on target tissue in a chamber of the heart with the procedural catheter while visualizing the target tissue using a technique selected from echocardiography, fluoroscopy, and intravascular ultrasound.

2. The method of claim 1, wherein the procedural catheter is advanced through the mediastinum under visualization using a technique selected from echocardiography, fluoroscopy, and intravascular ultrasound.

3. The method of claim 1, further comprising placing an endoscopic access device through the suprasternal penetration into the mediastinum, wherein the procedural catheter is advanced through the mediastinum in a working channel of the endoscopic access device.

4. The method of claim 1, further comprising positioning an access sheath through the suprasternal penetration into at least a portion of the mediastinum, the procedural catheter being advanced through a lumen of the access sheath.

5. The method of claim 4, wherein the access sheath is positioned through the atrial dome into the left atrium, the procedural catheter being advanced through the lumen of the access sheath into the left atrium.

6. The method of claim 1, wherein the interventional procedure is selected from mitral annuloplasty, chordal replacement, or mitral valve replacement.

7. The method of claim 1, wherein the interventional procedure comprises pulmonary vein ablation or atrial ablation.

8. The method of claim 1, wherein the interventional procedure comprises closure or occlusion of the left atrial appendage.

9. The method of claim 1, further comprising hemostatically sealing the puncture in the left atrial dome around the procedural catheter while performing the interventional procedure.

10. A method of performing an interventional procedure in a beating heart of a patient, the method comprising:
introducing an access catheter through a penetration at a suprasternal access site into a mediastinum of the patient;
advancing the access catheter through the mediastinum to an atrial dome of the heart with a sternum and ribs of the patient remaining intact;
advancing a tubular needle from an inner lumen of the access catheter to penetrate the atrial dome and extend into a left atrium of the heart;
inserting a guidewire through the needle into the left atrium;
removing the needle from the left atrium while leaving the guidewire extending through the inner lumen into the left atrium;
slidably advancing the access catheter over the guidewire into the left atrium;
removing the guidewire from the left atrium and the access catheter;
inserting a procedural catheter through the inner lumen of the access catheter into the left atrium; and
performing an interventional procedure on target tissue in a chamber of the heart with the procedural catheter, wherein the heart remains beating during the interventional procedure.

11. The method of claim 10, wherein a tubular dilator is positioned in the inner lumen of the access catheter as it is slidably advanced into the left atrium with the guidewire extending through the dilator.

12. The method of claim 10, wherein the interventional procedure is performed under visualization using a technique selected from echocardiography, fluoroscopy, and intravascular ultrasound.

13. The method of claim 10, wherein the access catheter is advanced through the mediastinum using a technique selected from echocardiography, fluoroscopy, and intravascular ultrasound.

14. The method of claim 10, wherein the interventional procedure is selected from mitral annuloplasty, chordal replacement, or mitral valve replacement.

15. The method of claim 10, wherein the interventional procedure comprises pulmonary vein ablation or atrial ablation.

16. The method of claim 10, wherein the interventional procedure comprises closure or occlusion of the left atrial appendage.

17. The method of claim 10, further comprising hemostatically sealing the puncture in the left atrial dome around the access catheter while performing the interventional procedure.

18. The method of claim 17, further comprising closing the puncture in the left atrial dome after the interventional procedure is performed.

19. The method of claim 18, wherein closing the puncture comprises delivering a suture through tissue of the atrial dome with a closure device positioned through the access catheter.
